(19) United States
(12) Patent Application Publication
(10) Pub. No.: US 2019/0062813 A1
(43) Pub. Date: Feb. 28, 2019

(54) SMART TOILET

(71) Applicant: Himanshu S. Amin, Solon, OH (US)

(72) Inventor: Himanshu S. Amin, Solon, OH (US)

(21) Appl. No.: 16/107,526

(22) Filed: Aug. 21, 2018

Related U.S. Application Data
(60) Provisional application No. 62/549,829, filed on Aug. 24, 2017.

(51) Int. Cl. C12Q 1/689; E03D 11/00; G06F 19/26; G06F 19/24; B01L 3/00

(52) U.S. Cl. CPC C12Q 1/689; E03D 11/00; G06F 19/26; B01L 2300/0636; B01L 3/5635; B01L 3/508; B01L 2300/0681; G06F 19/24

(57) ABSTRACT

Systems and methods disclosed herein relate to smart toilets (e.g. toilets with integrated stool analysis technology, urinalysis technology, deoxyribonucleic acid (DNA) sequencing technology, and/or logic circuitry which collectively enable the convenient, accessible, and real-time automation of waste analysis and microbiome screening). The smart toilet system can automatically perform various medical analyses on a user's biological waste, including DNA sequencing analyses, fecal occult blood tests, fecal pH tests, fecal fat tests, physical examinations of stool, particulate analyses of stool, physical analyses of urine, chemical analyses of urine, microscopic analyses of urine, particulate analyses of urine, and so on. The smart toilet system can then process and analyze the results in order to make appropriate diagnoses and/or to recommend various courses of action to the user so as to promote/preserve health.

### CROSS REFERENCE TO RELATED APPLICATION

This patent application claims the benefit of U.S. Provisional Application Ser. No. 62/549,829, filed on Aug. 24, 2017, the entirety of which is incorporated herein by reference.

### TECHNICAL FIELD

This disclosure relates generally to the field of analyzing human microflora. More particularly, this disclosure pertains to the integration of stool analysis technology, urinalysis technology, DNA sequencing technology, and/or logic circuitry into toilets to facilitate automated analysis and/or monitoring of human microflora.

### BACKGROUND

The human microbiome refers to the collective genetic material of trillions of microorganisms that live symbiotically in and/or on the various tissues and fluids of the human body and, most notably, the human gut. These myriad microorganisms (e.g., also referred to as microflora and/or microbiota) are intimately connected to overall bodily well-being and play important roles in regulating the immune system and digestive health. Recent studies have indicated possible correlations between gut microflora and various infirmities, such as gas, constipation, diarrhea, irritable bowel syndrome, diabetes, coronary artery disease, and even Parkinson's disease. Such findings have invigorated research and development efforts aimed at more fully understanding the human microbiome in the hopes of discovering new medical treatments.

With medical research hinting at the significant potential benefits of harnessing the human microbiome, many companies now offer microbiome screening tests (e.g., analyzing consumers' gut microflora). These conventional tests generally require an official order from an accredited health professional, manual collection of a biological waste sample by the patient, and/or mailing of the waste sample to the company performing the test. The company then analyzes the waste sample in a laboratory and may take several days, or even weeks, to return results to the patient. Moreover, since conventional waste analysis tests require manual collection and handling of stool and/or urine, the user is exposed to the risk of unwanted contact with human waste and the risk of accidentally spilling, contaminating, and/or otherwise mishandling the waste. Thus, there is a need in the field to provide more convenient, automated, and real-time (or near real-time) analysis of gut microflora and corresponding diagnoses and/or recommendations.

By incorporating, among other things, microbiome analysis technology into a toilet, one or more embodiments of the disclosed innovation can address the aforementioned deficiencies by obviating the act of receiving physician approval, by eliminating the need to manually collect and handle waste samples, and by not requiring mailing (or any other type of manual transportation) of the waste sample. Thus, various embodiments of the disclosed innovation can improve accessibility and convenience of microbiome screening/monitoring.

### SUMMARY

The following presents a simplified summary of the disclosure in order to provide a basic understanding of some aspects of the disclosure. This summary is not an extensive overview of the disclosure. It is intended to neither identify key or critical elements of the disclosure nor delineate any scope of particular embodiments of the disclosure, or any scope of the claims. Its sole purpose is to present some concepts of the disclosure in a simplified form as a prelude to the more detailed description that is presented later.

Systems, computer-implemented methods, and/or computer program products disclosed herein relate to a smart toilet (e.g., a toilet with integrated stool analysis technology, urinalysis technology, deoxyribonucleic acid (DNA) sequencing technology, and/or logic circuitry which collectively enable the convenient, accessible, and/or substantially real-time automation of waste analysis and microbiome screening/monitoring). In accordance with one or more embodiments, a system can comprise a processor (e.g., a central processing unit (CPU), a microcontroller, and so on) coupled to a computer-readable memory (e.g., a computer-readable storage medium, and so on). The processor can execute computer-executable instructions stored on the memory, thereby executing or facilitating execution of one or more computer-executable components. In one or more embodiments, the computer-executable components can comprise a filtration component that can automatically (e.g., not requiring manual handling by the user) collect, filter, and/or prepare at least one biological waste sample (e.g., stool, urine, vomit, menses, and so on) of a user from a toilet. In some embodiments, the filtration component can collect the sample actively, such as by a pump and/or propeller that, in response to execution by the processor, can actively draw in waste from the toilet, or by a mechanical manipulator and/or end effector that, in response to execution by the processor, can physically manipulate the user's waste (e.g., grabbing, slicing, scooping, catching, obtaining, acquiring, and so on). In some embodiments, the filtration component can collect the sample passively, such as by inlet and/or outlet apertures that can receive and/or expel the sample from and/or to the toilet during a flushing operation of the toilet. In various other embodiments, the filtration component can include a stationary fixture and/or apparatus on which, in which, by which, and/or through which the sample can be naturally deposited and/or separated as the sample enters the toilet bowl and/or as the sample is flushed away. In various embodiments, a combination of fixtures, structures, and/or apparatuses can be incorporated into the filtration component to facilitate the automated collection of the waste sample, wherein each fixture, structure, and/or apparatus can be particularly suited to collecting a certain type of waste (e.g., one collector suited for collecting stool, another suited for collecting urine, another suited for collecting vomit, and so on). One of ordinary skill in the art will appreciate that any means of automatically collecting a waste sample of the user from the toilet are in accordance with this disclosure.

In order to facilitate microbiome DNA screening/monitoring, the filtration component, in one or more embodiments, can further comprise a DNA extractor and/or a DNA amplifier. The DNA extractor can extract, isolate, and/or free the DNA contained within the microbial cells of the waste sample by physically and/or chemically homogenizing at least some of the sample (e.g., via chemical lysis, bead beating, centrifugation, blending, and so on). Then, the DNA amplifier can, if needed, amplify (e.g., replicate by several orders of magnitude) the extracted microbial DNA via polymerase chain reaction (PCR) methods, emulsion PCR methods, bridge amplification methods, and/or any other suitable amplification method known in the art.

In various embodiments, the executable components of the system can further comprise a waste analysis component that can, in response to execution by the processor, perform at least one medical and/or clinical test/analysis/examination on at least part of the waste sample. This test/analysis/examination can yield/produce raw data characterizing the sample (e.g., DNA sequence reads, results of automated chemical test strip exposures, results of automated visual/microscopic examinations, and so on), which raw data can subsequently be processed to make diagnoses and/or recommendations for the user.

To facilitate microbiome DNA analysis, the waste analysis component can, in various embodiments, comprise a DNA sequencer (e.g., an automated DNA sequencing device) that can receive the homogenized and/or amplified portion of a waste sample (or, in some embodiments, receive the raw sample itself without DNA extraction and/or amplification) and can automatically sequence the microbial DNA contained within the sample. In some cases, the DNA sequencer can also sequencer the user's own DNA. One of ordinary skill in the art will appreciate that any type of DNA sequencer can be implemented. For instance, in one or more embodiments, any method/technique of DNA sequencing known in the art and/or later created can be employed by the DNA sequencer, such as targeted amplicon sequencing (e.g., using the 16S rRNA genomic marker and/or other genomic markers), shotgun metagenomic sequencing, microbial metatranscriptomics, and so on. In various embodiments, the DNA sequencer can comprise suitable hardware and/or software to implement any known high-throughput sequencing method/technique (also known as massively parallel sequencing, next generation sequencing, and so on), such as ion semiconductor sequencing, pyrosequencing, polymerase-based sequence-by-synthesis, ligation-based sequencing, single molecule real-time sequencing (e.g., phospho-linked fluorescent nucleotide sequencing), dideoxy chain termination sequencing, nanopore sequencing, and so on. In some embodiments, the DNA sequencer can include suitable hardware and/or software to implement low-throughput sequencing methods (e.g., the longer runtimes of which can be offset by lower sequencing costs, and so on). Because different methods of automated DNA sequencing can have different costs, benefits, accuracies, and runtimes, the method chosen/implemented in a given embodiment can depend on a user's preferences and/or cost-benefit analysis. Ultimately, regardless of the sequencing technique used, the DNA sequencer can produce sequencing reads, which are raw data representing the order of the four nitrogenous bases (e.g., adenine, thymine, cytosine, and guanine) detected in at least a subset of the strands of microbial DNA present in the waste sample.

In one or more embodiments, the waste analysis component can further comprise a stool analyzer that can perform stool analysis on at least some of the waste sample. For example, the stool analyzer can, in various embodiments, comprise suitable hardware and/or software (e.g., an image capture device, a chemical test strip applicator, and so on) so as to implement a fecal occult blood test, a fecal pH test, a fecal fat test, a physical/visual stool examination, particulate/reducing substance analysis, and so on. Similarly, the waste analysis component can, in various embodiments, comprise a urine analyzer which can perform urinalysis on at least some of the sample. For instance, the urine analyzer can include suitable hardware and/or software (e.g., an image capture device, a chemical test strip applicator, and so on) so as to conduct a physical urine examination, a chemical examination, a microscopic examination, a particulate/reducing substance analysis, and so on.

In one or more embodiments, the executable components of the system can further comprise a processing component that can process and/or analyze the raw data produced by the waste analysis component. In various embodiments, the processing component can include a bioinformatics component which can analyze, interpret, and/or otherwise make sense of the sequencing reads produced by the DNA sequencer. In some embodiments, the bioinformatics component can have a sequence assembler, a reference database, and/or a sequence annotator. In such cases, the sequence assembler can utilize computational and/or statistical methods known in the art to assemble the various sequence reads produced by the DNA sequencer into separate, contiguous genomic segments (e.g., piecing together the disparate sequence reads produced by the DNA sequencer into contiguous genomes representing one or more microbial/microflora organisms living in the user's gut). These assembled genomic segments can then be compared to known genomic segments stored in the reference database in order to identify the microorganisms to which the assembled genomic segments belong. In this way, the various microflora of a user's gut can be identified. In some cases, the amount of a particular microbial organism living in the user's gut can be estimated based on the relative amount of the organism's DNA detected in the waste sample (and/or via other methods known in the art). Furthermore, if no known genomic segments match the assembled genomic segments, the sequence annotator can, in some embodiments, functionally annotate (e.g., identify various genes in a genome and determine what they do) the assembled genomic segments by comparing various portions of an unknown genome to portions of known genomes stored in the reference database, and/or by other methods known in the art. Said information can then be stored in the reference database for future use.

In various embodiments, the processing component can further include a stool analysis processing component and/or a urinalysis processing component which can perform similar post-test processing and/or analysis of raw data collected by the stool analyzer and the urine analyzer, respectively (e.g., visually analyzing an exposed reagent test strip to identify chemicals in the sample, implementing image/pattern recognition analyses to recognize particulates and/or parasites in captured images of the waste sample, and so on). In some cases, a single stool/urine analysis processing component can be implemented.

In one or more embodiments, the executable components of the system can further comprise a diagnostic component. The diagnostic component can, in some cases, leverage the processed results produced by the processing component to diagnose any diseases and/or infirmities which the user might have and/or to determine recommended courses of action for the user to take in order to remedy said diseases/infirmities and/or to otherwise preserve/maintain the user's health. Moreover, the diagnostic component can, in some cases, track the user's health and/or microflora composition over time (e.g., over multiple instances of the user using the smart toilet system), thereby creating a medical history of the user's gut health. This medical history can show how the microflora profile of a given user (e.g., the composition of the microbial population in the user's gut) has changed over time, thereby indicating the efficacy of past treatments/recommendations and offering insights for future treatments/recommendations. In some embodiments, the diagnostic component can progressively improve the accuracy of its diagnoses over time by performing subsequent analyses on the user's waste (e.g., performing multiple microbiome analyses on the user's waste over time and then piecing the various results together (e.g., via statistical and/or biological methods known in the art) to obtain a comprehensive and progressively more accurate profile of the user's microflora, and so on). In some embodiments, the diagnostic component can utilize artificial intelligence (AI) methods/techniques (e.g., machine learning, pattern recognition, inferential/probabilistic logic, and so on) in order to facilitate and/or enhance making diagnoses and/or recommendations to the user. Such AI capabilities can allow the diagnostic component to learn and account for any idiosyncrasies of the user (e.g., how the particular user responds to certain medicines and/or medical treatments as compared to the average human, and so on), and to thus tailor the diagnoses and/or recommendations to that particular user. In various other embodiments, the diagnostic component can run simulations of potential medical treatments, thereby predicting and/or approximating how the particular user's microflora profile might respond to a particular diet change, medical treatment, and so on. In some cases, the diagnostic component can be trained (e.g., explicitly and/or implicitly) with known waste samples and/or known recommended treatments to enhance the diagnostic component's inferential capabilities. In various embodiments, the diagnostic component can be configured to recommend/suggest commercial products (e.g., as a form of advertisement) to the user in order to address the diagnosed infirmity, and so on. Similarly, the diagnostic component can, in some cases, generate and/or identify (e.g., via the internet, and so on) recipes to cook and/or prepare recommended foods, routines and/or regimes for recommended exercises/actions, and so on.

In various embodiments, the executable components of the system can further comprise a notification component. The notification component can notify the user of the results of his/her microbiome screening (e.g., DNA analysis results, microbiome profile, stool analysis results, urinalysis results, and so on). The notification component can also inform the user of the determined diagnoses and/or recommendations (e.g., likely illnesses, suggested changes to diet, suggested exercises, suggested medicines, and so on). In various embodiments, the notification component can include a display and/or computer screen/monitor to notify the user. In other embodiments, the notification component can include a transmitter and/or transceiver so as to wirelessly send electronic notification messages to a device of the user (e.g., to the user's phone, personal digital assistant, laptop, desktop, smart television, smart dashboard display in a car, and so on). Moreover, the notification component can, in some embodiments, send microbiome analysis results (e.g., and/or any other results) to third parties (e.g., to medical professionals, to the user's friends and/or family, to researchers, to external programs and/or external AI systems, and so on), thereby facilitating a telemedicine aspect of the innovation. In some embodiments, medical professionals can receive the waste analysis results (e.g., in real time) and make the diagnoses and/or recommendations themselves. In various embodiments, the smart toilet system can notify a governmental body if particular chemicals are detected in the toilet water (e.g., if known poisons and/or toxins (such as lead) are detected (via chemical test strips), the smart toilet system can automatically notify an appropriate governmental authority that such poisons/toxins have likely been flushed into the public sewers and should promptly be addressed). In some cases, the notification component can notify the user of required maintenance and/or upkeep of the smart toilet system (e.g., inventory/maintenance sensors can be implemented in the smart toilet system and the notification component can then notify the user if the system is due for cleaning, if the chemical test strip inventory is low and/or needing replenished, if the fluorescent dye inventory is low and/or needing replenished, and so on). In various embodiments, the notification component can include an internet connection such that recommended foods, medicines, and/or commercial products can be automatically ordered online for the user. In some embodiments, the notification component can communicate (e.g., via a wireless and/or wired connection) with any smart appliances/devices of the user (e.g., preheating a smart over if the user desires to prepare a recommended food, inputting a store location into a GPS device of a smart vehicle so the user can purchase a recommended food/product, and so on).

In various embodiments, the computer-executable components can further comprise a user input component. The user input component can receive (e.g., via a receiver, transceiver, keyboard, touchscreen, microphone, and so on) user instructions (e.g., indicating whether to perform an analysis during this use of the smart toilet, and/or which analysis to perform, and so on), user feedback (e.g., whether prior diagnoses by the smart toilet system were verified or contradicted by medical professionals, whether prior recommendations improved the user's condition, and so on), idiosyncratic information (e.g., flavor/exercise preferences, medical history, lifestyle habits, allergies, and so on), and so on.

In some embodiments, the executable components can further comprise a user recognition component. In some cases, the user recognition component can automatically recognize the particular user using the smart toilet system (e.g., via voice recognition, weight recognition, appearance and/or image recognition, DNA recognition, and so on).

In various embodiments, the computer-executable components can further comprise a sterilization component. The sterilization component can, in some cases, automatically sterilize and/or otherwise clean the smart toilet system after and/or before analyzing a user's waste sample. This can allow the smart toilet system to analyze a waste sample from a different user with substantially reduced risk of cross-contaminating the first user's sample with the second user's sample. In one or more embodiments, the sterilization component can include pumps, propellers, and/or jets that rinse the smart toilet system with one or more disinfectants (e.g., ethanol, alcohol, bleach, soap, and so on). In some cases, the smart toilet system can be cleansed/sanitized by simply applying conventional cleaning techniques to the toilet.

In various embodiments, as mentioned above, DNA sequencing technology (and/or other waste analysis technology) can be integrated into a toilet (e.g., manufactured and/or built into the toilet itself). In one or more other embodiments, however, a modular housing affixable to a toilet bowl can include such waste analysis technology. The housing can comprise an inlet, a testing chamber, and an outlet. In some cases, waste from the toilet bowl can enter the inlet and flow into the testing chamber for the automated administration of a medical/clinical test (e.g., DNA analysis, stool analysis, urinalysis, and so on). In some cases, the waste can be drawn into the inlet and/or expelled through the outlet actively by a pump and/or propeller in the housing. In some cases, the inlet can passively receive (and the outlet can passively expel) the waste during a flushing operation of the toilet (e.g., the flushing operation can cause the toilet water and the waste it is carrying to flow into the inlet and through the outlet). The housing can further include a DNA sequencer (and/or any other automated waste analyzer) that can analyze the waste for microbial DNA, thereby producing at least one sequence read. In some embodiments, the housing can further include a transmitter and/or transceiver that can transmit the sequence read to a remote computing environment to process the sequence read (e.g., via bioinformatics).

The following description and the annexed drawings set forth certain illustrative aspects of the disclosure. These aspects are indicative, however, of but a few of the various ways in which the principles of the disclosure may be employed. Other aspects of the disclosure will become apparent from the following detailed description of the disclosure when considered in conjunction with the drawings.

### BRIEF DESCRIPTION OF THE DRAWINGS

FIG. 1 illustrates an exemplary, high-level methodology for analyzing human waste in accordance with one or more embodiments disclosed herein.

FIG. 2 illustrates an exemplary, high-level methodology for analyzing gut microflora via DNA analysis in accordance with one or more embodiments disclosed herein.

FIG. 3 illustrates an exemplary, high-level methodology for comprehensively analyzing human waste in accordance with various aspects disclosed herein.

FIG. 4 illustrates a high-level functional block diagram of an example, nonlimiting smart toilet system comprising various subcomponents in accordance with one or more embodiments disclosed herein.

FIG. 5 illustrates a high-level functional block diagram of an example, nonlimiting filtration component in accordance with one or more embodiments disclosed herein.

FIG. 6 illustrates a schematic of an example, nonlimiting waste collector in accordance one or more embodiments disclosed herein.

FIG. 7 illustrates a high-level functional block diagram of an example, nonlimiting waste analysis component in accordance with one or more embodiments disclosed herein.

FIG. 8 illustrates a high-level functional block diagram of an example, nonlimiting processing component in accordance with one or more embodiments disclosed herein.

FIG. 9 illustrates a high-level functional block diagram of an example, nonlimiting smart toilet system comprising an artificial intelligence component in accordance with one or more embodiments disclosed herein.

FIG. 10 illustrates a high-level functional block diagram of an example, nonlimiting smart toilet system comprising a user input component in accordance with one or more embodiments disclosed herein.

FIG. 11 illustrates a high-level functional block diagram of an example, nonlimiting smart toilet system comprising a user recognition component in accordance with one or more embodiments disclosed herein.

FIG. 12 illustrates a high-level functional block diagram of an example, nonlimiting smart toilet system comprising a sterilization component in accordance with one or more embodiments disclosed herein.

FIG. 13 illustrates a perspective schematic of an example, nonlimiting modular device affixable to a toilet bowl and that facilitates microbiome screening in accordance with one or more embodiments disclosed herein.

FIG. 14 illustrates an example block diagram of a computer operable to execute various implementations described herein.

FIG. 15 illustrates an example networking environment operable to execute various implementations described herein.

### DETAILED DESCRIPTION

The following detailed description is merely illustrative and is not intended to limit embodiments and/or application or uses of embodiments. Furthermore, there is no intention to be bound by any expressed or implied information presented in the preceding Background or Summary sections, or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

Systems, computer-implemented methods, and/or computer program products disclosed herein relate to smart toilets (e.g., toilets with integrated stool analysis technology, urinalysis technology, deoxyribonucleic acid (DNA) sequencing technology, and/or logic circuitry which collectively enable the convenient, accessible, and real-time automation of waste analysis and microbiome screening/monitoring). To thoroughly understand the disclosed innovation, consider the appended figures.

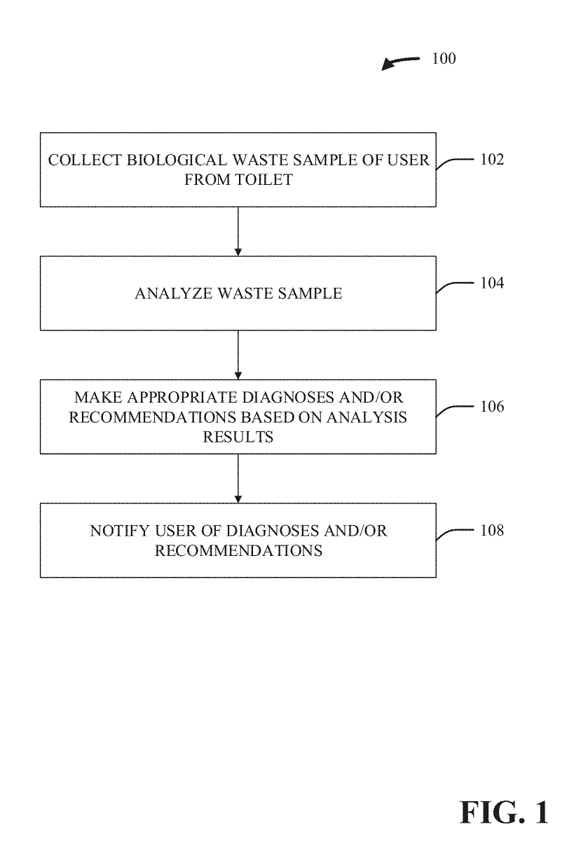

FIG. 1 illustrates an exemplary, high-level methodology for analyzing human waste in accordance with one or more embodiments disclosed herein. That is, FIG. 1 depicts a method 100 that demonstrates the high-level operation and functionality of one or more embodiments of the disclosed innovation.

At act 102, the smart toilet system can automatically collect a biological waste sample (e.g., stool, urine, vomit, menses, blood and so on) of a user from a toilet (e.g., as the user uses the toilet to defecate and/or urinate, and so on). As the user deposits waste into the toilet bowl, the smart toilet system can, in one or more embodiments, actively manipulate the waste to obtain/acquire at least one waste sample, such as by executable machinery which physically interacts with and/or affects the waste (e.g., a pump, a propeller, a water jet, a robotic end-effector, and so on). In various embodiments, the smart toilet system can passively collect waste samples as the waste enters the toilet and/or is flushed down the toilet (e.g., via inlet/outlet apertures, grates, ledges, dividers, tubes, sieves, and so on). In some embodiments, more than one collection apparatus/fixture can be employed such that a subset of collection apparatuses/fixtures is particularly suited to collecting at least one type of waste while another subset is particularly suited to collecting another type of waste (e.g., one collecting device/fixture that is particularly suited to collecting stool and another that is particularly suited to collecting urine, and so on). Furthermore, in some embodiments, a single collecting device/fixture can be used to collect any type of waste from the toilet bowl (e.g., an inlet that receives waste regardless of the type of waste, and so on).

At act 104, the smart toilet system can analyze the waste sample collected at act 102, thereby generating raw data characterizing the sample. As will be explained in detail below, various clinical and/or medical tests, analyses, and/or examinations can be performed at this act, including stool analysis (e.g., fecal fat test, fecal pH test, fecal occult blood test, particulate analysis, chemical analysis, visual/microscopic analysis, and so on), urinalysis (e.g., chemical analysis, ketone analysis, visual/microscopic analysis, and so on), microbial DNA sequencing analysis (e.g., identifying microflora organisms in user's gut based on detecting microbial DNA in the user's waste, and so on), and so on. Once the desired tests, analyses, and/or examinations are conducted, the raw data generated by said tests can be processed, yielding medically significant results (e.g., composition of the user's gut microflora, food recently eaten/consumed by the user, illnesses/infirmities of the user, and so on).

At act 106, the medically significant results can be leveraged to make appropriate diagnoses and/or recommendations to promote/preserve the user's health. For example, in some cases, act 104 can reveal that a user has an overabundance of undigested vegetable fibers in his/her stool (e.g., via an automated microscopic analysis using an image capture device, and so on). Since excessive vegetable fibers in stool can indicate insufficient chewing of food, the smart toilet system, at act 106, can determine that more thorough chewing is an appropriate course of action for the user. As another example, act 104, in some cases, can reveal the presence of fecal occult blood (e.g., via automatically exposing a chemical test strip to the sample and then visually analyzing the test strip via an image capture device, and so on), which cannot be detected by the naked eye and which can indicate upper and/or lower gastrointestinal bleeding. Since gastrointestinal bleeding can be caused by ulcers, gastrointestinal cancers, and/or other abnormalities, the smart toilet system can determine that the user should visit a doctor for proper intestinal screening. As yet another example, act 104 can reveal a dearth of a particular beneficial bacterium in the user's gut (e.g., via metagenomic DNA sequencing, and so on). So, at act 106, the smart toilet system can determine that eating more of one particular food and/or less of another can help to stimulate healthy levels of the bacterium in the user's digestive tract. In some cases, the diagnostic component's recommendations can be at any level of specificity/granularity, including how much of a recommended food to eat (e.g., number of grams and/or calories, and so on), how to prepare a recommended food (e.g., recipes, refrigeration, and so on), how to perform a recommended exercise and/or workout routine/regime (e.g., different types of cardio and/or weight lifting exercises, what order in which to perform the exercises, and so on), how much recommended exercise to perform (e.g., 30 minutes per day, no more than 35-pound dumbbells, and so on), and so on.

In some embodiments, the smart toilet system can track the results, diagnoses, and/or recommendations that it generates for a particular user over time. In such case, the smart toilet system can more accurately determine which diagnoses and/or recommendations seem to be efficacious (e.g., which recommended courses of action benefit the user the most) and which do not. In some instances, the smart toilet system can determine/estimate the level of efficaciousness of a recommendation by receiving user input and/or by comparing current waste analysis results with past results (e.g., comparing the user's current microflora profile with the user's past microflora profiles to determine how the user's microbiome has responded to past recommendations, and so on).

In some embodiments, the smart toilet system can piece/weave together (e.g., via statistical methods known in the art) multiple waste analyses performed at different times to generate more comprehensive and/or more accurate results. For instance, the results of a microbial DNA analysis can be artificially skewed for any number of reasons (e.g., the sample was insufficiently homogenized, the user's last meal was uncharacteristic of his/her usual diet, and so on). In such case, a first DNA analysis of the user's waste can reveal an overabundance of microorganism L because the waste sample was insufficiently homogenized/lysed and not because the microorganism L actually exists at such elevated levels in the user's gut. Subsequent analyses (e.g., over the next several days and/or weeks, and so on) can reveal more normal levels (and/or underabundant levels) of microorganism L. The smart toilet system can use statistical and/or other methods known in the art to combine these various results to provide a comprehensive and collectively more accurate microflora profile of the user's gut (e.g., taking the average estimated amounts of microorganism L over all the analyses rather than solely relying on a single, possibly outlying analysis; creating a confidence interval based on these multiple analyses; and so on).

As explained at length below, artificial intelligence (AI) techniques (e.g., machine learning, inferential logic, probabilistic reasoning, and so on) can be implemented at act 106 to enhance the efficacy of the diagnoses and/or recommendations (e.g., learning the user's idiosyncratic health aspects, adjusting current and/or future treatment recommendations based on whether prior treatment recommendations were effective, and so on). For instance, in some embodiments, act 106 can leverage idiosyncratic information about the user (e.g., learned via user input, via previous diagnoses made by the smart toilet system, via analyzing the user's own DNA, and so on) to improve the efficacy of diagnoses and/or recommendations. Such idiosyncratic information can include, but is not limited to, height of the user, weight of the user, body-mass-index of the user, age of the user, temperature of the user, ethnicity of the user, lifestyle habits of the user (e.g., smoking, drinking, and so on), geographic location of the user, family medical history of the user, allergies of the user, previously diagnosed health conditions of the user, currently prescribed and/or over-the-counter medications taken by the user, flavor/exercise preferences of the user, and so on. Such idiosyncratic information can be leveraged by the smart toilet system (e.g., via AI functionalities) to help diagnose any infirmities suffered by the user and/or to recommend restorative courses of action.

As an example, act 104 can, in some cases, reveal that the user has a suboptimal amount of Lactobacillus bacteria (e.g. a type of beneficial microorganism) in his/her gastrointestinal tract. The smart toilet system can know and/or learn (e.g., via artificial intelligence methods/techniques, via connection to the Internet, via access to a medical/clinical database, via explicit/implicit training, and so on) that increased consumption of almonds can correspondingly increase Lactobacillus populations in human digestive tracts. However, if the user is allergic to almonds (e.g., which the smart toilet system can learn via receiving user input, via analyzing the user's own DNA, and so on), the smart toilet system can refrain from recommending that the user increase his/her consumption of almonds, despite the fact that almonds would generally be beneficial to the average person in such case. Instead, the smart toilet system can recommend an alternative food source to stimulate Lactobacillus growth (e.g., yogurt, and so on). Moreover, the smart toilet system's diagnoses and/or recommendations can be based on the user's age, height, and/or weight (e.g., to determine appropriate amounts of recommended foods, exercises, and/or medicines), familial history of the user (e.g., to determine if the user is genetically predisposed to suffering from particular diseases/infirmities), any current medications taken by the user (e.g., to refrain from recommending foods/products that could negatively interact with the user's medications), any previous diagnoses made by medical professionals pertaining to the user (e.g., to refrain from recommending foods, products, and/or exercises that could exacerbate the user's pre-existing condition and/or to avoid contradicting official medical diagnoses), preferences of the user (e.g., to refrain from recommending foods that the user finds un-flavorful and/or exercises/actions that the user finds too strenuous and/or unenjoyable), and so on.

In some embodiments, the smart toilet system can learn particular idiosyncratic information about the user without requiring the user to manually input such information. For instance, the smart toilet system can leverage a timeseries of the user's microflora profile (e.g., examining/analyzing multiple microflora profiles of a given user, each profile representing the state of the user's microflora at a particular instant in time) to learn that the user's microflora consistently have an underabundance of organism X (e.g., a particular beneficial microorganism) and/or an overabundance of organism Y (e.g., a particular harmful microorganism), and so on. The smart toilet system can then base its recommendations on this learned information (e.g., refraining from recommending a particular food/product if healthy levels of microorganism X are needed to digest/metabolize it, refraining from recommending a particular exercise/action if healthy levels of microorganism Y are needed to perform it, and so on). In some embodiments, the smart toilet system can analyze the user's own DNA (e.g., via DNA sequencing and/or annotating methods described below) to uncover and/or learn any genetic abnormalities afflicting the user. Such abnormalities/quirks can then be taken into account during the diagnosing and/or recommending acts, and so on.

Prior waste analyses performed by the smart toilet system on the particular user can, in one or more embodiments, be leveraged to improve the efficacy of current diagnoses and/or recommendations. To continue the above example, the user having low levels of Lactobacillus can increase his/her consumption of yogurt as suggested by the smart toilet system. When the user subsequently uses the smart toilet system (e.g., after some time of eating more yogurt), the smart toilet system at act 104 can again determine that the user's Lactobacillus levels are still suboptimal. The smart toilet system can learn (e.g., via user input, via particulate analyses, and so on) that the user properly increased consumption of yogurt, as suggested previously, and can also learn and/or track how much time elapsed between the previous microflora screening and the current screening. If an insufficient amount of time has elapsed (e.g., not enough time for the increased consumption of yogurt to noticeably improve the user's condition), the smart toilet system can determine that it is too early to tell whether the treatment is working and/or can recommend that the increased consumption of yogurt be maintained and/or increased further. If, on the other hand, a sufficient amount of time has elapsed (e.g., more than enough time for the increased consumption of yogurt to noticeably improve the user's condition), the smart toilet system can determine that the increased yogurt consumption alone is not working and can recommend an alternative and/or complementary course of action (e.g., consume more fermented/pickled vegetables which can help to stimulate healthy Lactobacillus levels, and so on).

In some embodiments, the smart toilet system can run computational simulations (e.g., statistical regressions, agent-based models, and so on) regarding potential recommended courses of action (e.g., medical treatments, lifestyle changes, and so on). Such simulations can help to estimate and/or predict how effective such courses of action can be for the user. In some cases, such simulations can take into account the inputted and/or learned idiosyncrasies of the user (e.g., age, height, weight, ethnicity, family medical history, lifestyle habits, allergies, current medications, pre-existing conditions, genetic dispositions, preferences, and so on) as well as any available medical data regarding how different (and/or average) members of the population react to particular treatments (e.g., Center for Disease Control data, World Health Organization data, local hospital data, and so on). In some embodiments, the smart toilet system can receive (e.g., via wired and/or wireless connections, and so on) information from other smart toilet systems (and/or any other type of smart medical device) regarding how such other users responded to recommended treatments; thus, the smart toilet system can learn (and thereby improve its diagnostic and recommendation capabilities) from the collective experience of other smart toilet systems. In some cases, the smart toilet system can learn how to better diagnose and/or recommend solutions for a particular user by examining its success (and/or lack thereof) in diagnosing and/or treating that same user in the past and/or in diagnosing and/or treating different users in the past. In some cases, the smart toilet system can be explicitly and/or implicitly trained to provide proper/appropriate diagnoses and/or recommendations (e.g., given known samples to analyze, shown which recommended treatments work best for given samples, and so on).

In various embodiments, the recommendations of the smart toilet system can be based on commercial and/or proprietary products, foods, and/or medicines. For instance, the smart toilet system at act 104 can determine that the user lacks an optimal amount of fiber in his/her diet (e.g., via automated chemical and/or physical examination of waste, and so on). In such case, the smart toilet system at act 106 can be configured to specifically recommend that the user consume more Fiber One® bars, rather than generally recommending higher fiber intake. In such case, the smart toilet system can function as a form of product advertisement, and so the manufacturers of such proprietary products, medicines, and/or foods can compensate a manufacturer of the smart toilet system for such advertisement accordingly.

Finally, at act 108, the smart toilet system can notify the user of the diagnoses and any recommended courses of action to promote/preserve the user's health. In various embodiments, this can be accomplished by any means suitable for notification of a user. For instance, the smart toilet system can notify (e.g., via wired connections, wireless transmitters and/or transceivers, and so on) the user by means of a visual message and/or alert composed of text and/or numbers that is received and/or displayed on a laptop computer, a desktop computer, a mobile device, dedicated computer displays/monitors that are locally and/or remotely connected to the smart toilet system, any other device capable of displaying such visual messages/alerts, and/or any combination thereof. In other embodiments, the smart toilet system can notify by means of an audible message and/or alert that is received and/or played by a laptop computer, a desktop computer, a mobile device, dedicated speakers/microphones that are locally and/or remotely connected to the smart toilet system, any other device capable of playing such audible messages/alerts, and/or any combination thereof. In still other embodiments, the smart toilet system can notify by means of vibratory messages and/or alerts, in much the same way that mobile devices vibrate when they receive text messages or emails. Furthermore, in some cases, any combination of the aforementioned notification methods can be incorporated into the smart toilet system. Those of skill in the at will appreciate that any other method/technique of notifying a user of analysis results can be incorporated (e.g., flashing and/or lit up lights, and so on). Moreover, one of ordinary skill in the art will appreciate that any level of detail can be conveyed in the notification. For example, various embodiments of the disclosed innovation can equally include a notification which conveys the exact numeric values produced by the DNA and/or other waste analyses as well as a notification which indicates merely that a user's gut health is good or bad.

In some embodiments, the smart toilet system at act 108 can send the waste analysis results, diagnoses, and/or recommendations to entities other than the user (e.g., remote medical professionals, remote family members and/or friends, remote researchers, extrinsic programs and/or external AIs, and so on). Thus, the smart toilet system can be implemented in a telemedicine fashion, allowing the user to conveniently share his/her results with others and/or to receive diagnoses, recommendations, and so on from others (e.g., via a receiver, and so on).

In some embodiments, the smart toilet system can communicate (e.g., via wired and/or wireless connections) with any other smart appliances of the user (e.g., smart phone, smart TV, smart kitchen, smart oven, smart car, and so on). For instance, if the smart toilet system determines that the user should eat more of a particular food, the smart toilet system can, in some cases, communicate with a smart oven of the user, so as to preheat the smart over in order to help the user prepare the recommended food/meal. As another example, the smart toilet system can, in some embodiments, communicate with a smart vehicle, so as to input a grocery store address into the vehicle's GPS device in order to help the user travel to the store to purchase a recommended food/product. In one or more embodiments, the smart toilet system can order products from the internet for the user. For instance, if the user does not currently possess a recommended food/product (e.g., learned via user input, learned via communication with a smart refrigerator and/or cupboard inventory manager, and so on), the smart toilet system can automatically search for the food/product online and/or place an online order for the food/product. In some cases, the smart toilet can have access to the user's payment and/or delivery information (e.g., credit card number, home address, and so on). In other cases, the smart toilet system can request user input (e.g., user can manually input such payment/delivery information) so as to place the order.

In various embodiments, the smart toilet system can notify the user of required and/or recommended cleaning, maintenance, and/or upkeep of the smart toilet system. That is, maintenance and/or upkeep sensors can be installed in the smart toilet, and the smart toilet can notify the user (and/or others) when the sensors indicate that upkeep is needed. For example, if the smart toilet system requires manual cleaning (e.g., via a toilet brush and/or conventional toilet cleaners, and so on), the user can be notified that such cleaning is recommended to prevent contaminating a current waste sample with remnants of a previous waste sample (e.g., which contamination could result in inaccurate DNA analyses, and so on). As another example, the smart toilet system can notify the user if any waste analysis materials in the smart toilet system need to be repaired and/or replenished (e.g., chemical test strip inventory is low and/or needs replenished, lysis buffer reservoir is low and/or needs replenished, DNA sequencer is damaged and/or otherwise needs to be repaired, and so on).

In some cases, the smart toilet system can notify an appropriate governmental entity if poisons and/or toxins are detected in the toilet bowl/water (e.g., if a chemical test strip indicates that an overabundance of lead is in the toilet bowl, the smart toilet system can notify a governmental body that such levels of lead are being flushed into the public sewers, and so on).

In one or more embodiments, the method 100 can be implemented by a toilet that has integrated stool/urine analysis and/or DNA analysis technology. In other embodiments, as described below, the method 100 can be implemented in a modular device that is affixable to a toilet bowl and that houses stool/urine analysis and/or DNA analysis technology.

In various embodiments, the total runtime of methodology 100 can range from real-time and/or near-real-time (e.g., nearly instantaneous analysis and results) to hours, days, or even weeks, depending on the technology and/or analytic techniques incorporated into the smart toilet system. For example, regarding microbial DNA sequencing, nanopore sequencing can display results in real-time, dideoxy chain termination sequencing can require between twenty minutes and a few hours to run, and ligation-based sequencing can take several weeks to complete. Thus, real-time or near-real-time analysis results are possible, which allows various embodiments of the disclosed innovation to be more convenient than conventional waste analysis and microbiome screening/monitoring.

Moreover, the disclosed innovation can provide other significant benefits. First, since one or more embodiments integrate high-functionality electronics and technology into a standard toilet, users can perform a full waste analysis and receive the corresponding results from the comfort of their own bathrooms. The fact that complete microbiome analyses can be conducted without even leaving one's own home underscores the improved convenience and accessibility of the disclosed innovation over traditional microbiome testing apparatuses and methods. It is to be appreciated that the location of the smart toilet system is immaterial (e.g., it can be located in a private residence, in a place of work, in a hospital, in a clinic, on a vehicle, in an outhouse, and so on). Second, since the disclosed innovation involves automated waste collection and analysis, the user is not bothered with manual collection, handling, and/or transportation of stool and/or urine (or any other biological waste). Accordingly, the user is not exposed to the risk of unwanted contact with waste samples, and the probability of accidental sample spills, contaminations, and/or other mishandlings (as well as the likelihood that the user contracts an illness due to exposure to waste) can be eliminated. These benefits are not merely abstract; rather, they constitute notable technological improvements in the field of automated waste analysis and microbiome screening.

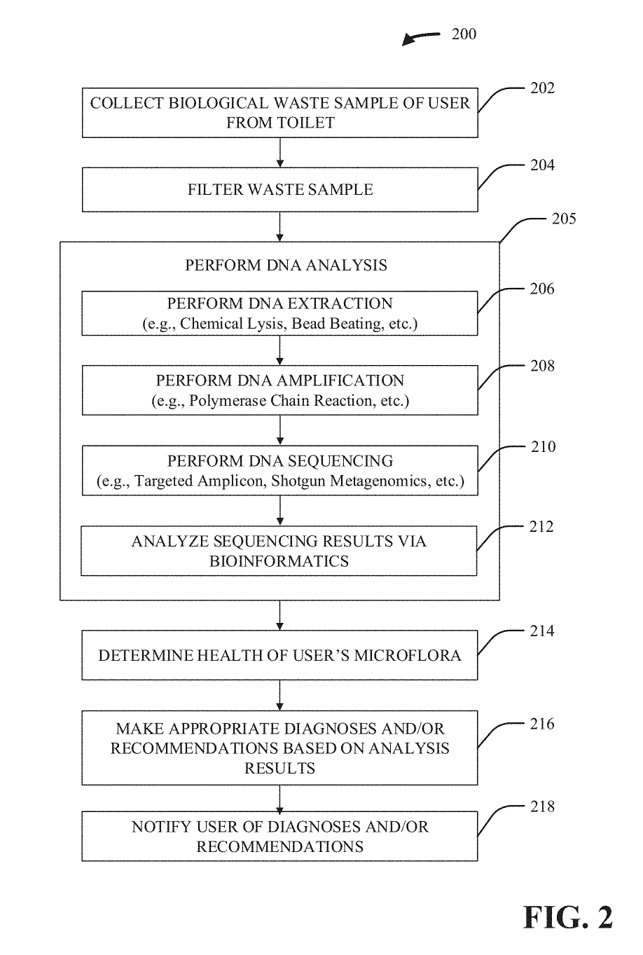

Now, consider FIG. 2. FIG. 2 illustrates an exemplary, high-level methodology for analyzing gut microflora via DNA analysis in accordance with one or more embodiments disclosed herein. That is, FIG. 2 depicts a method 200 that facilitates automated DNA analysis and microflora screening of a biological waste sample.

At act 202, the smart toilet system can collect a biological waste sample (e.g., stool, urine, vomit, and so on) of a user from a toilet. As mentioned above in conjunction with act 102 of method 100, the waste collection can be performed actively and/or passively by appropriate, executable and/or non-executable apparatuses, fixtures, and/or apertures, and so on.

At act 204, the smart toilet can, in some embodiments, filter the waste sample (e.g., separating solid waste from liquid waste, and so on). Generally, stool analysis and urinalysis are performed separately to avoid cross-contamination. Thus, if the smart toilet system collects a sample comprising both stool and urine (e.g., or any other combination of biological waste), filtering at act 204 can be performed (e.g., via grates, drains, and/or sieves, so as to drain the collected urine away from the collected stool, and so on). In some embodiments, the smart toilet system, at act 202, can collect multiple separate samples (e.g., one stool sample and one urine sample, and so on) so as to avoid the cross-contamination issue altogether. In such case, waste collection and filtration can be combined into a single act. In various embodiments, filtering and/or otherwise separating the waste sample into different types of waste can be omitted (e.g. a waste sample of any composition can be automatically collected at act 202 and subsequently analyzed without separating/filtering the sample into stool and urine portions (and so on) at act 204).

At act 205, the smart toilet system can perform a DNA analysis on the waste sample (e.g., a metagenomic DNA analysis to determine the composition of the user's gut microflora, and so on). In various embodiments, act 205 can be decomposed into constituent acts 206-212, as shown. At act 206, the smart toilet system can, in some embodiments, perform DNA extraction on the collected waste sample. DNA extraction, also known as DNA isolation and/or sample homogenization, is a form of cellular disruption (e.g., cell lysis), which is the process of breaking down microbial cell walls and/or membranes so as to release (e.g., in an aqueous solution) the biologically significant molecules contained within the microbes (e.g., DNA, RNA, proteins, and so on).

As known in the art, cellular disruption can be performed in aqueous solution chemically and/or physically. Chemical lysis can involve chemically breaking down the cell walls and membranes of the collected sample and can be accomplished by the use of lysis buffers, which can comprise surfactants/detergents (which can partition membrane proteins from cell membranes), lytic enzymes (which can free sub-cellular contents from cell wall envelopes), and chaotropic agents (which can disrupt the ordered structure of biological macromolecules by affecting intramolecular interactions), and so on. Physical lysis, on the other hand, can involve the mechanical homogenization of the collected sample and can be accomplished by the use of bead beating, centrifugation, mechanical blending/shearing, microfluidizers, cryopulverization, and so on. Bead beating can involve suspending the sample in aqueous media, adding appropriately-sized, hard beads, and then cyclically agitating the solution at a high frequency, thereby causing the beads to shear the cell walls and membranes of the sample. Similar physical agitation can be achieved via centrifuges and/or mechanical blenders (e.g., shaking, stirring, mixing, and/or cutting an aqueous solution of the sample to homogenize it, and so on). Microfluidizers can accomplish similar shearing by using hydraulic pressure to force suspended samples through fixed microchannels, thereby generating high shear forces which can break cell walls and membranes. Finally, cryopulverization can involve cooling a sample to liquid nitrogen temperatures and then crushing the sample into a fine powder, thereby rupturing the cells in the sample. One having ordinary skill in the art will appreciate that other methods and/or devices for cellular disruption are possible and in accordance with this disclosure. Moreover, any combination of the aforementioned cellular disruption methods and/or other methods known in the art may be used concurrently and/or in series in accordance with this disclosure.

In one or more embodiments, once the sample is properly lysed, the lysate (e.g., the solution containing the contents of the lysed cells) can be refined by adding further surfactants/detergents (e.g., to break down lipids from the cell membranes and nuclei), proteases (e.g., to break down proteins released by the cellular disruption), and RNase (e.g., to break down RNA released by the cellular disruption). The lysate can then, in some cases, be purified by centrifugation to separate the microbial DNA (e.g., the biological molecules of interest) from the broken down cellular material (e.g., proteins, RNA, cell walls, cell membranes, lipids, and so on). One of ordinary skill in the art will appreciate that any other method of cellular disruption and/or purification is in accordance with this disclosure. In various embodiments, the various surfactants, detergents, and/or aqueous solutions needed to implement DNA extraction can be contained within actuatable reservoirs in the smart toilet system (e.g., via actuated valves, and so on).

In various embodiments, a flushing operation of the toilet can sufficiently homogenize the sample (e.g., the swirling of the toilet water can cause the waste to collide and/or break down in the toilet water, and a sample of that toilet-water-and-waste solution can be received/collected by the smart toilet system and examined/analyzed directly). In such case, an explicit DNA extraction act can be omitted.

At act 208, the smart toilet system, in one or more embodiments, can perform DNA amplification on the lysed sample. DNA amplification is the process by which small amounts of sample DNA are cloned, creating thousands to millions of copies of the DNA sequences of interest. This can be accomplished by any suitable method of DNA amplification known in the art, such as polymerase chain reaction (PCR), and so on.

As is known in the art, basic PCR amplification can involve the use of a thermostable DNA polymerase (e.g., an enzyme which polymerizes new strands of DNA), two oligonucleotide primers (e.g., short, known strands of DNA that bind to the 3′ ends of target DNA, thereby providing an initiation site to which the polymerase can bind), deoxynucleotide triphosphates (e.g., nucleotides with triphosphate groups which are used by the DNA polymerase to synthesize new DNA strands), a buffer solution (e.g., an aqueous solution which maintains a nearly constant pH value to ensure optimum performance of the DNA polymerase), bivalent cations (e.g., ions with a positive charge and a valency of two), and monovalent cations (e.g., ions with a positive charge and a valency of one). The above-mentioned reactants can be assembled in the reaction chamber of a thermal cycler, wherein each thermal cycle (e.g., repeated heating and cooling) can consist of three main acts: denaturation; primer annealing; and/or primer extension. At the denaturation act, the thermal cycler can sufficiently heat the reaction chamber, thereby breaking the hydrogen bonds between the complementary nitrogenous bases of the double-stranded target DNA and causing it to separate into two strands. At the primer annealing act, the temperature of the reaction chamber can be lowered, allowing the oligonucleotide primers to attach to the separated strands of target DNA. Finally, at the primer extension act, the thermal cycler can heat and/or cool the reaction chamber to the optimum temperature for the DNA polymerase, allowing the polymerase to synthesize new DNA using the deoxynucleotide triphosphates. The cycle can be repeated as often as required to achieve suitable amplification.

In one or more embodiments, each act of the cycle can take several seconds to a couple minutes to complete, and tens of cycles can be required, thereby yielding an amplification time ranging from minutes to hours. Although this act can add time to the waste analysis, it does not require the user to wait days and/or weeks to receive his/her results, thereby still enabling near-real-time microbiome analysis.

Moreover, as is known in the art, other variations of PCR amplification exist, such as emulsion PCR, bridge PCR, quantitative PCR, hot-start PCR, reverse transcription PCR (for the sequencing of RNA rather than DNA), long PCR, and so on, as well as entirely different methods of DNA amplification. One having ordinary skill in the art will appreciate that implementing any of these methods with the disclosed innovation is in accordance with this disclosure.

In various embodiments, any buffers and/or solutions required to perform DNA amplification can be stored in actuatable reservoirs in the smart toilet system (e.g., via actuated valves, and so on).

Those of ordinary skill in the art will further appreciate that, in one or more embodiments, act 206 and/or act 208 can be omitted (e.g., the collected sample can be analyzed/examined directly without having to perform explicit DNA extraction and/or amplification, and so on).

At act 210, the smart toilet system, in an embodiment, can perform DNA sequencing on the extracted, amplified, and/or raw DNA of the sample, thereby generating data characterizing the sample (e.g., generating at least one DNA sequence read associated with the sample). DNA sequencing is the process of analyzing a strand of DNA to determine the precise order of the four nitrogenous bases (e.g., adenine, thymine, cytosine, and guanine) in that strand. In one or more embodiments, this act can be carried out by an automated DNA sequencer known in the art. More broadly, any suitable method of DNA sequencing may be incorporated into various embodiments of the disclosed innovation, such as targeted amplicon sequencing, shotgun metagenomic sequencing, and so on.

Targeted amplicon sequencing can refer to the ultra-deep sequencing of PCR products (e.g., amplicons) to search for genetic variation in a specific genomic region. One of the most common genetic markers (e.g., a specific genomic region) used in targeted amplicon sequencing is the 16S rRNA gene, which is the small subunit ribosomal gene that has been highly conserved in all DNA-based life forms. The 16S rRNA gene can comprise both conserved and variable genetic regions, which makes it very useful for identifying complex microbial communities. Specifically, the conserved regions can enable the use of universal primers during PCR amplification (which allows analysis of the DNA of a large diversity of organisms), while the variable regions, once sequenced, can be used to distinguish between different microorganisms.

Shotgun metagenomic sequencing, on the other hand, can refer to the sequencing method comprised of randomly breaking up long DNA sequences into many short fragments, sequencing those fragments (which can be much quicker than sequencing the original sequences themselves), and then determining the original sequences by using overlapping regions in the fragment reads to assemble the reads together. Shotgun metagenomic sequencing can thus allow extremely long and/or numerous DNA sequences to be analyzed, and can thus be used to sequence entire genomes as opposed to searching only for specific genetic markers. Shotgun metagenomic sequencing can and/or cannot require PCR amplification, depending on the sequencing technology used.

Regardless of the DNA sequencing method used in a given embodiment, one having ordinary skill in the art will appreciate that any suitable DNA sequencing technology can be incorporated into one or more embodiments of the disclosed innovation, such as massively parallel sequencing platforms (also known as next-generation sequencing or high-throughput sequencing) or traditional low-throughput sequencing platforms. Examples of massively parallel sequencing technologies known in the art include ion semiconductor sequencing, pyrosequencing, polymerase-based sequence-by-synthesis, ligation-based sequencing, single molecule real-time sequencing (also known as phospho-linked fluorescent nucleotide sequencing), dideoxy chain termination sequencing, nanopore sequencing, and so on.

Ion semiconductor sequencing is one form of sequencing by synthesis, in which a single strand of target DNA can be placed in a microwell along with a DNA polymerase and sequentially flooded with one species (e.g., one of adenine, thymine, cytosine, or guanine) of deoxynucleotide triphosphate. If the species of deoxynucleotide triphosphate is complementary (e.g., adenine complements thymine, and cytosine complements guanine) to the leading unpaired nucleotide of the target DNA strand, it can be incorporated into the target strand, thereby forming a covalent bond and releasing a pyrophosphate and a positive hydrogen ion. The hydrogen ion can then trigger an ion sensor (e.g., an ion-sensitive field-effect transistor). If the deoxynucleotide triphosphate is not complementary to the leading unpaired nucleotide of the target strand, no reaction occurs. Once the results of adding one deoxynucleotide triphosphate are determined (e.g., reaction or no reaction), the previous deoxynucleotide triphosphate can be washed out of the microwell and a new one can be added. In this way, the nucleotide bases of a given strand of target DNA can be determined. The advantages of ion semiconductor sequencing include rapid sequencing speeds (approximately two hours per run) and inexpensive operating costs. One limitation of ion semiconductor sequencing is increased difficulty in identifying homopolymer repeats of the same nucleotide in a target DNA strand (e.g., when one nucleotide is repeated several times in a row). When a given nucleotide is repeated in a target strand, multiple hydrogen ions can be emitted when sufficiently many complementary deoxynucleotide triphosphates bind to the target DNA, thereby increasing the intensity of the signal reported by the ion sensor. This clearly indicates repeated nucleotides, but the exact number can be difficult to determine.

Pyrosequencing is another method of sequencing by synthesis. Just as with ion semiconductor sequencing, different species of deoxynucleotide triphosphate can be sequentially added to a single strand of target DNA. However, pyrosequencing can involve determining the amount of resulting pyrophosphate instead of the amount of hydrogen ions released by the reaction. To accomplish this, a single strand of target DNA can be hybridized to a sequencing primer and incubated with DNA polymerase. The enzymes adenosine triphosphate (ATP) sulfurylase, luciferase, and apyrase can be added to the incubation, as can be the substrates adenosine 5′ phosphosulfate (APS) and luciferin. Once a deoxynucleotide triphosphate complementary to the leading nucleotide on the target strand is added, the deoxynucleotide triphosphate can bind to the strand, thereby releasing pyrophosphate and a hydrogen ion. The ATP sulfurylase and APS together can convert the pyrophosphate to ATP. The ATP, in conjunction with the luciferase, can then convert the luciferin to oxyluciferin, thereby releasing visible light wherein the amount of light can be proportional to the amount of ATP. The generated light can be captured by a camera and/or other image capture device and analyzed; the very fact that light appears at all indicates the identity of the nucleotide in the target strand while the amount of light can indicate how many repeated nucleotides there are. Finally, the apyrase can degrade any ATP and unincorporated deoxynucleotide triphosphate, allowing the cycle to repeat. Pyrosequencing can be generally slower than ion semiconductor sequencing (e.g., requiring approximately twenty-four hours), can be unideal for sequencing long strands of target DNA, and can suffer from the same limitation concerning homopolymer repeats as ion semiconductor sequencing. However, pyrosequencing can boast improved DNA sequencing accuracy.

Polymerase-based sequence-by-synthesis (also known as Illumina dye sequencing) is another method of sequencing by synthesis. After DNA extraction, tagmentation can be performed, wherein the DNA can be randomly cut into short segments via transposome enzymes and then ligated (e.g., outfitted) with adapters. Next, primers, indices, and terminal sequences can be added to the DNA segments, wherein these additions can function as molecular markers and allow the target DNA to bind to stationary oligonucleotides to facilitate sequencing. The target DNA can then be amplified by methods known in the art. After amplification, primers can attach to the target strands, thereby adding fluorescently tagged nucleotides to the target DNA. A camera and/or other image capture device can then analyze the wavelength of the light emitted by the fluorescent tag of the added nucleotide to determine which base was added, the complement of which is the nucleotide of interest in the target DNA. Non-incorporated molecules can then be washed away and the cycle can be repeated. Advantages of polymerase-based sequence-by-synthesis include the fact that expensive enzymes, such as those used in pyrosequencing, are not required. Limitations of polymerase-based sequence-by-synthesis include slow runtimes (e.g., on the order of days).

Ligation-based sequencing, unlike the above-mentioned sequencing methods, does not involve sequencing by synthesis (e.g., identifying target bases by adding one known nucleotide at a time). Rather, ligation-based sequencing can rely on the enzyme DNA ligase, which can preferentially bind fluorescently-tagged, partially-degenerate oligonucleotide strands (e.g., eight to nine bases in length, and so on) to target DNA only when there is not base-pair mismatch. First, a single strand of target DNA can be flanked by a strand with a known sequence. Then, an anchor strand, which complements the known strand, can bind to the known strand. Partially degenerate oligonucleotides (e.g., single strands with at least one known nucleotide at a known position) can be fluorescently labeled and mixed with the target DNA. DNA ligase can then preferentially join the anchor strand to an oligonucleotide whose bases complement the target DNA. Imaging equipment can be used to analyze the fluorescent tag of the hybridized oligonucleotide, thereby identifying the known nucleotide of the oligonucleotide. The nucleotide of the target DNA is simply the complement of this known nucleotide. Optionally, the fluorescent labels can be cleaved, allowing the next oligonucleotide to be ligated, thereby identifying another nucleotide of the target sequence that is located eight or nine bases away. Once the end of the target DNA strand has been reached, the process can be repeated with an anchor strand of different length, thereby enabling the identification of other target nucleotides. Ligation-based sequencing can be quite slow (e.g., on the order of weeks) but is easily implemented and highly accurate.

Single molecule real-time sequencing can return sequencing data in real-time by observing the activity of a single DNA polymerase on a single strand of target DNA within a zero-mode waveguide. A zero-mode waveguide can be an optical waveguide (e.g., a structure designed to guide electromagnetic waves in the optical spectrum) which creates a volume all of whose dimensions are very small (e.g., on the order of tens of nanometers) as compared to the wavelength of light. The volume can be illuminated and can be used to observe the incorporation of individual nucleotides by a DNA polymerase. Inside the zero-mode waveguide, a single strand of target DNA can be immobilized and an observation chamber (e.g., the volume) can be created which is large enough to see only the activity of a DNA polymerase with respect to a single nucleotide. Each available nucleotide can be tagged with a fluorescent marker. A detector monitoring the observation volume of the zero-mode waveguide can identify the fluorescent tag of the nucleotide incorporated into the target strand by the DNA polymerase, thereby identifying the added nucleotide and its complement. As the synthesis proceeds, the fluorescent tag can be cleaved off and is no longer detected. The next nucleotide added by the DNA polymerase can then be observed. Advantages of single molecule real-time sequencing include quick (e.g., approximately two to four hours) and inexpensive runtimes. Moreover, this method does not require DNA amplification, further saving time and resources. However, single molecule real-time sequencing can be generally less accurate than other sequencing methods.

Dideoxy chain termination sequencing (also known as Sanger sequencing) can involve terminating DNA synthesis with a known nucleotide to identify the target nucleotide at the terminal base. This method can require a single strand of target DNA, a DNA primer, a DNA polymerase, standard deoxynucleotide triphosphates (dNTPs), and dideoxynucleotide triphosphates (ddNTPs), wherein the ddNTPs lack a 3′-OH group required to continue DNA synthesis and are fluorescently or radioactively tagged. Target DNA can be fragmented into small pieces and then amplified by methods known in the art. DNA synthesis can then be initiated, wherein extension can continue if a dNTP is incorporated into the fragment strand and can cease if a ddNTP is incorporated into the fragment strand. The result is myriad terminated fragments of different lengths. These fragments can be separated via electrophoresis, and their nucleotides can be determined by identifying the radioactive or fluorescent tags of their terminal bases. These reads can then be assembled to determine the overall target sequence. This method generally boasts speedy runtimes (e.g., on the order of minutes to hours), long read lengths, and very high accuracy.

Finally, nanopore sequencing is a method of real-time sequencing that, unlike the above-mentioned methods, does not require either amplification or fluorescent tagging. There are two types of nanopore sequencing: solid-state sequencing and biological sequencing. Solid-state sequencing can incorporate the use of various metals while biological sequencing can use transmembrane proteins. In either case, the method can involve using electrophoresis to transport a DNA strand through a nanopore (e.g., a hole with a diameter on the order of nanometers that is composed of either metal or proteins). An electrolytic solution can be integrated into the nanopore system such that a constant electric current is measured across the nanopore surface. This electric current can change depending on the composition of the DNA inside the nanopore, such that each of the four different nitrogenous bases distinctly affects the electric current. By measuring the electric current across the nanopore surface, the identity of the nucleotide passing through it can be determined. Nanopore sequencing is relatively new, returns results in real time, and has been integrated into hand-held machinery that is much smaller than the machinery required for other methods of DNA sequencing. However, nanopore sequencing also can have a comparatively low accuracy rate.

One having ordinary skill in the art will appreciate that other sequencing technologies and/or platforms are known in the art and are thus in accordance with this disclosure. Furthermore, one of ordinary skill will appreciate that automated versions of the above-mentioned DNA sequencing techniques as well as any other methods known in the art have been developed and can be implemented in various embodiments of the disclosed innovation. Moreover, any other type of DNA sequencing method and/or device that is later created in the art can be incorporated in various embodiments.

At act 212, the smart toilet system can, in one or more embodiments, computationally analyze and/or process the raw data produced by the DNA sequencing act via bioinformatics tools and/or methods known in the art. Bioinformatics leverages mathematics, statistics, chemistry, biology, and/or computer science in order to analyze vast amounts of data obtained through biological experiments. In various embodiments, the smart toilet system can assemble the voluminous, fragmentary sequence reads produced by DNA sequencing analysis (e.g., act 210) to reconstruct at least one whole sequence associated with the waste sample. This can be accomplished computationally using any sequence assembly software and/or hardware known in the art (e.g., automated sequence assemblers). Moreover, one having ordinary skill in the art will appreciate that the method of assembly (e.g., expressed sequence tag assembly, de-novo assembly, mapping assembly, and so on) is immaterial, as all such methods are in accordance with various embodiments of this disclosure.

Once at least one whole sequence is determined (either through sequence assembly or by the DNA sequencing act itself), it can be compared to known DNA sequences, which can be stored in local or remote databases, for identification. For example, the DNA sequencing and processing acts can return a sequence that corresponds to the known sequence of a certain bacterium (or virus, parasite, and so on), thereby indicating that the bacterium is present in the user's microflora. The smart toilet can further determine whether the amount of said DNA detected in the waste sample corresponds to a healthy population of said bacterium in the user's gut (e.g., estimating population size of the bacterium in the user's gut based on the detected amount of the bacterium's DNA in the analyzed sample, and so on) and can recommend various courses of action accordingly (e.g., dietary changes, exercise changes, medical advice, and so on).

If no sequence matches are identified, the smart toilet system can, in one or more embodiments, structurally and/or functionally annotate the at least one whole DNA sequence. Genetic annotation can involve computationally identifying various genes and/or other coding regions in a DNA sequence and determining what they do. This can be accomplished through automatic annotation software tools known in the art (e.g., automated DNA annotators). For instance, the smart toilet system can determine that no known DNA sequence matches a sequence obtained from the waste sample through DNA sequencing analysis. Then, the smart toilet system can perform genetic annotation via automated annotation software to determine that various portions of the obtained sequence correspond to genetic segments known to code for the production of a certain biological product or for the performance of a certain biological function. In such case, the smart toilet system can infer that microorganisms known to create said biological product or to perform said biological function are present in the user's microflora and can notify the user accordingly.

At act 214, the smart toilet system can, in various embodiments, determine the health of the user's microflora. This can be accomplished by comparing and/or contrasting the obtained DNA sequence data (e.g., both the genetic information contained within a DNA sequence and/or the abundance/amount of a particular DNA sequence within the sample) with reference data representing an average, healthy microflora ecosystem (e.g., an average person's gut). Such reference data can be obtained from medical/clinical databases (e.g., Center for Disease Control data, World Health Organization data, local hospital data, and so on). For example, the smart toilet system, at act 212, can determine the identities of various microorganisms living within the user's gut (e.g., by matching obtained DNA sequences to known DNA sequences) as well as the relative populations of those microorganisms within the user's gut (e.g., by comparing the amount of one particular DNA sequence collected from the sample to another, wherein a more abundant DNA sequence in a sample can indicate a more abundant corresponding organism in the user's gut). The smart toilet system can then compare this information with reference information representing a healthy microflora ecosystem to conclude that the user has too much and/or too little of one or more particular microorganisms within his/her gut. Furthermore, the smart toilet system can determine whether the types and/or amounts of certain microflora within the user's gut correspond to known infirmities (e.g., irritable bowel syndrome, Parkinson's disease, lactose intolerance, allergies, and so on). In some cases, artificial intelligence capabilities can be used to improve this act.

At act 216, the smart toilet system, in one or more embodiments, can make appropriate diagnoses and/or recommendations based on the analysis results in order to promote and/or preserve the user's health, such as foods to avoid, foods to eat, exercises/actions to perform, exercises/actions to avoid, medicines to take, medicines to avoid, and so on (e.g., substantially as described above). For example, the smart toilet system can determine that the user has too much of microorganism X and not enough of microorganism Y. Then, the smart toilet system can determine (e.g., via AI capabilities) that the user should eat more of food A and/or less of food B in order to bring his/her microflora back into balance.

Finally, at act 218, the smart toilet system can notify the user of any diagnoses and/or recommendations made at act 216 (e.g., substantially as described above). As explained above, any method of notification is in accordance with various embodiments of the disclosed innovation. Moreover, telemedicine can be implemented by sharing the user's results with remote medical professionals, and so on.

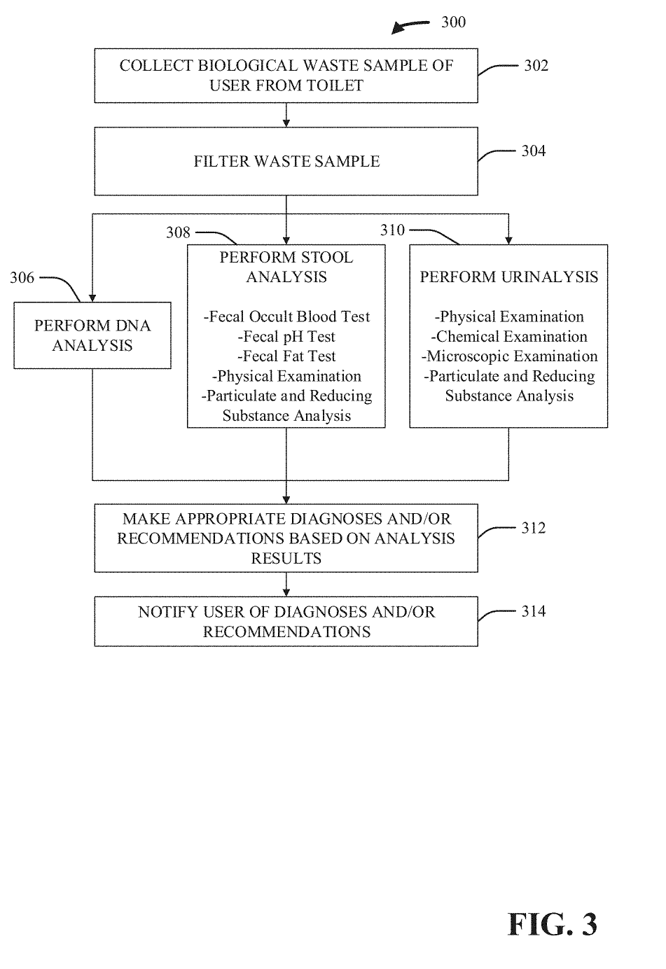

Now, consider FIG. 3. FIG. 3 illustrates an exemplary, high-level methodology for comprehensively analyzing human waste in accordance with various aspects disclosed herein. That is, FIG. 3 depicts a method 300 that facilitates performing a DNA analysis, a stool analysis, and/or a urinalysis (and/or another other type of analysis) on a waste sample of a user, which sample is automatically collected from a toilet.

At act 302, the smart toilet system can collect a biological waste sample (e.g., stool, urine, vomit, and so on) from a user, just as explained above. At act 304, the smart toilet system can, in some embodiments, filter the waste sample (e.g., into solid and/or liquid portions, and so on), also as explained above. At act 306, the smart toilet system can, in various embodiments, perform DNA sequencing analysis (amply explained above in connection with FIG. 2) on at least part of the sample.

In one or more embodiments, the smart toilet system can perform additional types of waste analysis, such as automated stool analysis at act 308 and/or automated urinalysis at act 310.

Stool analysis can include any type of automated medical test that is performed on a user's stool, such as a fecal occult blood test, a fecal pH test, a fecal fat test, a physical stool examination, a particulate analysis, a visual/microscopic examination, a chemical test strip analysis, and so on.

Fecal occult blood refers to blood in stool that cannot be detected by the naked eye. The presence of fecal occult blood can indicate upper and/or lower gastrointestinal bleeding, which can be caused by ulcers, gastrointestinal cancers, and/or other abnormalities. In various embodiments, the smart toilet system can comprise any method known in the art to detect fecal occult blood, such as by fecal immunochemical testing (which detects globin in stool by the use of specialized antibodies), stool guaiac testing (which detects heme in stool by the use of hydrogen peroxide), chemical/reactive test strips (e.g., plastic and/or paper dipsticks having chemical pads that react with particular chemicals to produce characteristic colors, wherein the resulting colors of the pads indicate the identities of the tested chemicals), and so on.

Fecal pH (e.g., acidity versus alkalinity) can indicate general intestinal health. Since human stool is usually alkaline, acidic feces can indicate digestive issues such as lactose intolerance, rotavirus, E. coli, fat malabsorption, disaccharidase deficiency, carbohydrate malabsorption, and so on. However, an overly alkaline stool may indicate colitis, villous adenoma, diarrhea, and so on. In various embodiments, the smart toilet system can comprise any method known in the art by which fecal pH is measured, such as through the use of chemical/reagent test strips, nitrazine paper, and so on.

Fecal fat testing is a diagnostic tool for detecting steatorrhea (e.g., fat malabsorption). Since fat is a relatively valuable nutrient, very little is normally found in human feces. If excess fat is detected, it can be indicative of pancreatitis, cystic fibrosis, celiac disease, and so on. In one or more embodiments, the smart toilet system can comprise any method known in the art by which fecal fat is detected, such as by Sudan staining and automated microscopy, and so on.

A physical/visual stool examination can include any method known in the art by which a stool sample is grossly and/or microscopically examined. Gross examinations can include examination of the stool for consistency (e.g., loosely formed, watery, thin, pellet-like, dry/hard, and so on), color (e.g., brown can indicate normal, gray can indicate ingestion of chocolate and/or steatorrhea, black can indicate ingestion of iron and/or bleeding of the upper gastrointestinal tract, dark brown can indicate a high meat diet, red can indicate a diet high in beats and/or bleeding of the lower gastrointestinal tract, green can indicate a high vegetable diet, and so on), quantity (e.g., indicates how well a user digests their food), odor (e.g., can indicate excessive consumption of carbohydrates and/or undigested lactose), mucous (e.g., can indicate severe constipation, mucous colitis, ulcerative colitis, and even emotional instability), and so on. Microscopic examinations can include automated visual examination of the stool for leukocytes (e.g., white blood cells, the presence of which can indicate bacillary dysentery, ulcerative colitis, shigellosis, salmonella, a fistula of the anus or rectum, typhoid, and so on), red blood cells (e.g., can indicate bleeding in the gastrointestinal tract caused by hemorrhoids, cancer, dysentery, and so on), parasites and/or ova (e.g., can indicate that the user is infected with roundworms, hookworms, pinworms, whipworms, tapeworms, and so on), fat (e.g., can indicate malabsorption, bile deficiency, pancreatic enzyme deficiency, and so on), meat and muscle fibers (e.g., can indicate cystic fibrosis, and so on), and so on. Such analyses can be performed by any suitable method and/or apparatus known in the art, such as by automated imaging equipment (e.g., automated microscopes and/or cameras with magnified lenses, and so on), automated microscopy equipment, electronic noses, staining/dying methods, chemical/reactive test strip applicators (e.g., inventories of chemical test strips and/or actuatable end-effectors that can expose a chemical test strip from the inventory to the sample), electronic weight sensors/scales, and so on.

In some cases, the smart toilet system can perform any other form of particulate/reducing substance analysis on at least part of the sample so as to identify foods consumed, fat content, salt content, fiber content (both muscle fibers and vegetable fibers), carbohydrate content, elastase content, short chain fatty acid content (e.g., percent acetate, percent propionate, percent butyrate, percent valerate, and so on), inflammatory substance content (e.g., lactoferrin, calprotectin, lysozyme, and so on), and so on. Said analyses can be performed by any suitable method and/or apparatus known in the art, such as by automated imaging equipment, automated chemical test strip applicators, staining/dying methods, automated microscopy, and so on.

Similarly, urinalysis can include any type of medical test that is conventionally performed on a user's urine, such as a physical examination, a chemical examination, a microscopic examination, a particulate analysis, and so on.

A physical examination of urine can include any method known in the art by which a urine sample is examined for color (e.g., colorless, light yellow, dark yellow, amber, and so on, where the darker colors can indicate diets high in carrots, beets, rhubarb, other vegetables, or certain drugs), transparency (e.g., clear, hazy, cloudy, turbid, and so on, where higher opaqueness can indicate the presence of phosphates, urates, mucous, bacteria, and so on), specific gravity (e.g., where low specific gravity can indicate diabetes insipidus, excessive hydration, chronic renal failure, and so on, and high specific gravity can indicate diabetes mellitus, excessive dehydration, kidney inflammation, and so on), mucous, and so on. Such analyses can be performed by any method and/or apparatus known in the art, such as by automated imaging equipment, electronic noses, refractometers, urinometers, weight sensors/scales, chemical test strip applicators, and so on.

A chemical examination of urine can include any method known in the art by which a urine sample is examined for pH (e.g., abnormally high acidity can indicate excessive ingestion of protein or bacterial infections while abnormally high alkalinity can indicate excessive ingestion of vegetables), proteins (e.g., such as albumin, the presence of which can indicate pregnancy, fatigue, high protein diets, bacterial toxins, and so on), glucose (e.g., can indicate diabetes mellitus, and so on), ketones (e.g., such as acetoacetic acid, beta-hydroxybutyric acid, and acetone, the presence of which can indicate diabetes mellitus, starvation, diarrhea, and so on), bilirubin (e.g., arising from the breakdown of hemoglobin, the presence of which can indicate liver disorders, cirrhosis, hepatitis, obstruction of a bile duct, and so on), urobilinogen (e.g., arising from the breakdown of hemoglobin, the presence of which can indicate hemolytic anemia, liver disease, and so on), hemoglobin (e.g., can indicate hemolytic anemia, blood transfusion reactions, renal disease, and so on), red blood cells (e.g., can indicate kidney stones, tumors, glomerulonephritis, trauma, and so on), white blood cells (e.g., can indicate a urinary tract infection, and so on), nitrite (e.g., can indicate a urinary tract infection, and so on), and so on. Such analyses can be performed by any suitable method and/or apparatus known in the art, such as by automated chemical/reagent test strips, automated staining/dying methods, image capture devices, and so on.

A microscopic examination of urine can include any method known in the art by which a urine sample is visually examined for epithelial cells (e.g., can indicate kidney disease, a urinary tract infection, and so on), leukocytes (e.g., can indicate a urinary tract infection, an obstruction of the urinary tract and/or bladder, kidney stones, tumors, and so on), erythrocytes (e.g., can indicate kidney disease, a blood disorder, bladder cancer, and so on), renal cells (e.g., can indicate tubular necrosis, heavy metal intoxication, and so on), crystals (e.g., such as uric acid, triple phosphate, calcium oxalate, and so on, the presence of which can indicate kidney stones, gout, high protein diets, a urinary tract infection, ingestion of antifreeze, cystinuria, liver disease, and so on), yeast (e.g., can indicate a urinary tract infection, and so on), bacteria (e.g., can indicate bladder, urethra, or kidney infections, and so on), urinary casts (e.g., can indicate dehydration, vigorous exercise, renal disease, kidney disease, nephritic syndromes, renal infarction, systemic lupus erythematosus, inflammation, toxin ingestion, and so on), and so on. Such analyses can be performed by any suitable method and/or apparatus known in the art, such as by automated centrifugation and microscopy, automated imaging equipment, computational imaging analysis, and so on.

Finally, just as explained above in connection with stool analysis, the smart toilet system can, in various embodiments, perform any particulate/reducing substance analysis for urine, via any suitable method and/or apparatus known in the art.

At act 312, the smart toilet system can make appropriate diagnoses and/or recommendations based on the results of the analyses at acts 306-310, just as explained above. Then, at act 314, the smart toilet system can notify a user of said diagnoses and/or recommendations, as previously explained.

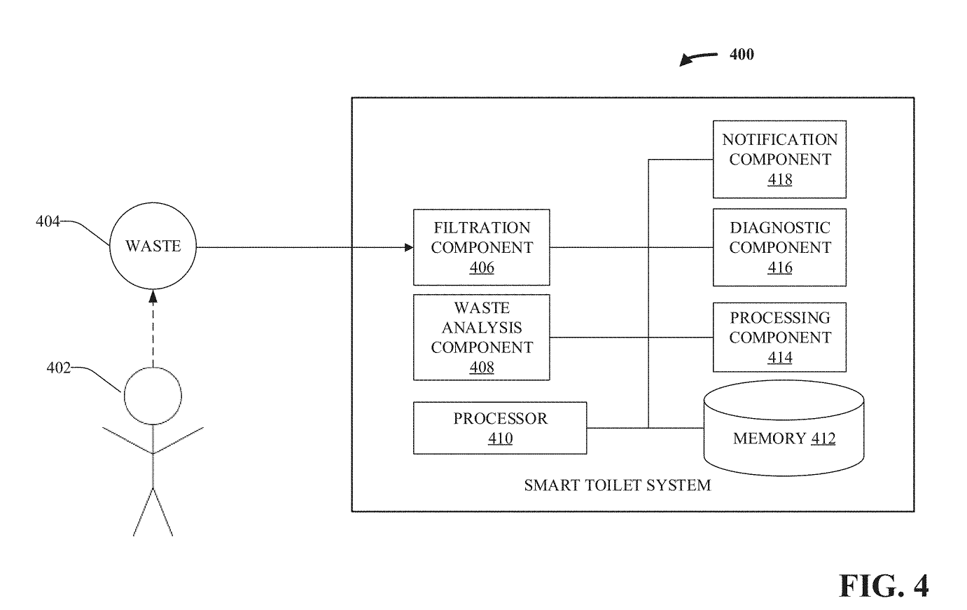

Now, consider FIG. 4. FIG. 4 illustrates a high-level functional block diagram of an example, nonlimiting smart toilet system comprising various subcomponents in accordance with one or more embodiments disclosed herein. That is, FIG. 4 depicts a smart toilet system 400 that can, in various embodiments, facilitate/implement one or more of the methodologies 100, 200, and/or 300.

In one or more embodiments, the smart toilet system 400 can comprise an electronic processor 410 and a computer-readable memory 412, wherein the processor 410 can execute and/or facilitate execution of computer-executable instructions stored on the memory 412, thereby executing and/or facilitating execution of one or more computer-executable components (e.g., the other subcomponents of the smart toilet system 400).

As shown, a user 402 can produce biological waste 404 (e.g., stool, urine, vomit, and so on). The waste 404 can be automatically collected by a filtration component 406 (e.g., during and/or after the waste 404 enters a toilet bowl (not shown in FIG. 1) and/or as the waste 404 is flushed away). As explained above, the filtration component 406, in various embodiments, can actively manipulate the waste to obtain at least one appropriate sample, such as by executable machinery which can physically interact with the waste 404. For example, the filtration component 406 can comprise one or more executable, robotic manipulators/end-effectors which can touch, push, pull, move, grab, scoop, slice, and/or otherwise actively interact with the waste 404 in response to execution, thereby collecting a sample of the waste 404. Such manipulators can, in various embodiments, comprise movable claws and/or arms, movable surfaces of the toilet bowl, water/air jets and/or pumps that push/pull waste into intake/outtake apertures, and so on. In one or more embodiments, the filtration component 406 can passively collect a waste sample (e.g. during a flushing operation of the toilet, as the waste enters the toilet, and so on). For instance, the filtration component 406 can be outfitted with stationary fixtures, such as inlet/outlet apertures, grates, ledges, dividers, tubes, hooks, stationary claws, and so on. In some cases, a flushing operation of the toilet can cause the toilet water (and the waste 404 that the toilet water is carrying) to swirl, and this swirling motion can cause an inlet to receive the waste 404. In yet other embodiments, a combination of the aforementioned collection methods/apparatuses/fixtures (and/or any others known in the art) can be used. Furthermore, the filtration component 406 can, in some aspects, comprise more than one apparatus/fixture for collecting a waste sample, such that one subset of said apparatuses/fixtures is particularly suited to collecting stool while another is particularly suited to collecting urine, and so on. Moreover, if the filtration component 406 collects a single sample of waste 404 comprising both stool and urine, the filtration component 406 can, in some embodiments, separate the stool from the urine, such as by draining the urine into a separate sample container, and so on. In some cases, a single inlet/outlet channel can be configured to receive any type of waste 404 (e.g., solid waste, liquid waste, and so on); in such case, separation/filtering of the solid/liquid portions of the waste can be omitted.

After collecting a waste sample, the filtration component 406 can, in some embodiments, perform filtration and/or preparation of the samples. Such filtration and/or preparation can include, for example, DNA extraction (e.g., via chemical lysis and/or mechanical homogenization) and DNA amplification (e.g., via any PCR and/or other method known in the art). In some cases, the smart toilet system 400 can separate the collected stool and/or urine into sufficiently many testable samples and can perform any other necessary and/or beneficial filtration and/or preparation of said samples (e.g., centrifugation, stirring, mixing, straining, heating, cooling, dying, staining, storing, and so on).

Once the sample is collected and/or prepared by the filtration component 406, it can be received by the waste analysis component 408. The waste analysis component 408 can, in one or more embodiments, perform DNA sequencing analysis on the sample. The waste analysis component 408 can, in various embodiments, comprise suitable hardware and/or software so as to automatically implement any suitable DNA sequencing method (e.g., targeted amplicon sequencing and/or shotgun metagenomic sequencing) and platform (e.g., ion semiconductor sequencing, pyrosequencing, polymerase-based sequence-by-synthesis, ligation-based sequencing, single molecule real-time sequencing (also known as phospho-linked fluorescent nucleotide sequencing), dideoxy chain termination sequencing, nanopore sequencing, and so on) known in the art, as explained thoroughly above. To facilitate this, automated DNA sequencers as known in the art can be incorporated into the waste analysis component 408.

After conducting the DNA sequencing analysis, the waste analysis component 408 will have generated data characterizing the sample (e.g., at least one sequence read which represents the order of the four nitrogenous bases in at least some DNA sequence detected in the sample). These reads can then be processed in order to help determine the health of the user's microflora.

In some embodiments, the waste analysis component 408 can comprise suitable hardware and/or software so as to automatically implement any other type of waste analysis (e.g., stool analysis, urinalysis, and so on). As explained above, such analyses can include fecal occult blood tests, fecal pH tests, fecal fat tests, physical/visual (e.g., gross and/or microscopic) examinations of stool/urine, particulate/reducing substance analyses of stool/urine, chemical/reactive test strip analyses of stool/urine, and so on. In various embodiments, such analyses can be accomplished through the use of automated imaging equipment and computational image/pattern recognition software, automated staining/dying equipment (e.g., Sudan staining), automated microscopy equipment, electronic noses, automated chemical/reagent test strip applicators, other suitable methods/apparatuses known in the art, and so on. The results of such analyses can then be processed, if needed, in order to help determine the digestive health of the user.

After the waste analysis component 408 performs at least one medical/clinical test, the resulting raw data can be received by the processing component 414. As explained above, the processing component 414 can analyze and/or process the raw data yielded by the waste analysis component 408 in order to convert the raw data into medically significant results (e.g., converting raw sequence reads into whole DNA sequences to identify microorganism organisms in the user's gut, using chemical/reagent test strip colors to identify target chemicals in the sample, using captured images of waste to identify particulates and/or structures in the sample, and so on). In some embodiments, the processing component 414 can comprise suitable hardware and/or software so as to implement bioinformatics methods known in the art in order to assemble (e.g., through de novo assembly, mapping assembly, and so on) the fragmentary DNA sequence reads produced by the waste analysis component 408 into at least one contiguous DNA sequence, as explained above. The contiguous DNA sequences can then be compared and contrasted with known DNA sequences stored in local and/or remote databases. By comparing the obtained DNA sequences to known DNA sequences, the various microorganisms comprising the microflora ecosystem of the user's gut can be identified. Furthermore, by comparing the relative amounts of the different DNA sequences identified in the sample with each other, the relative proportions of the identified microorganisms living in the user's gut can be estimated. This information can then be used to determine whether the user's microflora is balanced or imbalanced.

If no known DNA sequences match an obtained DNA sequence, the processing component 414 can structurally and/or functionally annotate the at least one obtained DNA sequence. As explained above, genetic annotation involves computationally identifying various genes and/or other coding regions in a DNA sequence and determining what these coding regions do (e.g., code for the production of a hazardous biological product, code for the performance of a certain biological function, and so on). This can be accomplished through automatic annotation software tools known in the art. Thus, even if the smart toilet system 400 is unable to identify a particular microorganism detected in a testable sample, the processing component 414 can, in various embodiments, determine what the microorganism can do and can thus classify the microorganism as beneficial, pathogenic, nonpathogenic, and so on.

In other embodiments, the processing component 414 can comprise any suitable hardware and/or software needed for the proper processing and/or analysis of raw data produced by automated stool analysis and/or urinalysis. In some embodiments, the processing component 414 can comprise automated image/pattern recognition software so as to analyze an image produced by the waste analysis component 408. For example, the waste analysis component 408 can use automated imaging equipment to capture a microscopic image of a waste sample. Then, the processing component 414 can analyze the captured image in order to detect notable structures, such as parasites/ova, urinary casts, meat/vegetable fibers, red blood cells, white blood cells, epithelial cells, crystals, and so on. As another example, the waste analysis component 408 can use automated chemical/reagent test strip applicators (e.g., dipsticks) to test the sample for certain chemical properties (e.g., pH, occult blood, proteins, glucose, ketones, and so on). Since such chemical tests generally involve the use of chemically treated dip sticks which change color depending on the type and/or quantity of target chemicals in the sample, the processing component 414 can use imaging software to compare the colors yielded by the dip sticks to reference colors indicating known types and/or quantities of target chemicals. One with ordinary skill in the art will appreciate that any other type of processing known in the art that is required and/or helpful to convert the raw data produced by the waste analysis component 408 into medically significant results is in accordance with this disclosure.

Once the raw data characterizing the sample is processed to yield medically significant results (e.g., determining identities and/or amounts of certain microbiota in the user's gut, identifying pathogenic and/or concerning structures and/or chemicals in the user's waste, and so on), a diagnostic component 416 can leverage the medically significant results to determine and/or evaluate the user's health. For instance, the diagnostic component 416 can, based on the processed data, diagnose any infirmities/diseases suffered by the user (e.g., too few of microorganism X can indicate disease Y, too much of microorganism A can indicate allergy B, and so on) and/or recommend courses of action to the user in order to preserve and/or maintain the user's health (e.g., eat more of food M to stimulate healthy levels of organism N, take medicine O in order to reduce amount of chemical P, and so on). As explained above, the processed data can, in one or more embodiments, indicate identified microorganisms, relative proportions of identified microorganisms, presence/amount of fecal occult blood, fecal pH value, fecal fat content, composition of various particulate matter identified in stool/urine, presence/amount of various chemicals in stool/urine, consistency of stool/urine, odor and/or color of stool/urine, and so on. The diagnostic component 416 can, in some cases, compare this medically significant information with known reference values (e.g., representing averages and/or healthy persons) to evaluate the user's health.

For example, the processing component 414 can produce data indicating that a user has X units of microorganism A in his/her gut. So, the diagnostic component 416 can access a local and/or remote database to learn the fact that an average user has Y units of microorganism A in his/her gut. Then, the diagnostic component 416 can determine that the user has too much or too little of microorganism A in his/her gut, depending on the values of X and Y, and diagnose a corresponding medical condition. Furthermore, the diagnostic component 416 can determine recommended solutions for promoting/preserving health by accessing a local and/or remote medical/clinical database to determine how such an imbalance in microorganism A is usually cured. In some cases, the diagnostic component 416 can be explicitly and/or implicitly trained (e.g., shown which recommendations best resolve which infirmities/conditions, and so on).

As another example, the processing component 414 can produce data indicating that a user has M units of chemical J in his/her waste. So, the diagnostic component 416 can access a local and/or remote database to learn the fact that an average user has N units of chemical J in his/her waste. Then, the diagnostic component 416 can determine that the user has too much or too little of chemical J in his/her waste, depending on the values of M and N, and diagnose a corresponding medical condition. Furthermore, the diagnostic component 416 can determine recommended solutions for promoting/preserving health by accessing a local and/or remote database (or by being trained) to determine how such a condition is usually cured.

Moreover, the granularity of the reference values utilized by the diagnostic component 416 is immaterial. For instance, various embodiments of the smart toilet system 400 can equally encompass comparing a user's processed results with those of an average, healthy person as well as comparing a user's processed results with those of an average, healthy individual of the same age, gender, size, weight, height, BMI as the user, and so on.

Once diagnoses and/or recommendations are made, a notification component 418 can notify the user of the diagnoses and/or recommendations (and/or any other results of the waste analysis). As explained above, any method/device for notifying a user can be incorporated into various embodiments of the disclosed innovation (e.g., wired connections to dedicated monitors/speakers, wireless connections (via transmitters or transceivers) to dedicated monitors/speakers, visual and/or audio messages sent to any device of the user (e.g., computer, desktop, laptop, mobile phone, radio, smart car, smart kitchen, and so on), and so on). As explained above, the notification component can include an internet connection (e.g., wired or wireless) to send notifications to non-users (e.g., remote medical professionals, remote researchers, family, and so on), to order recommended foods/products for the user online, to search for information online in order to make and/or bolster the diagnoses and/or recommendations of the diagnostic component 416, to communicate with other smart devices of the user, and so on.

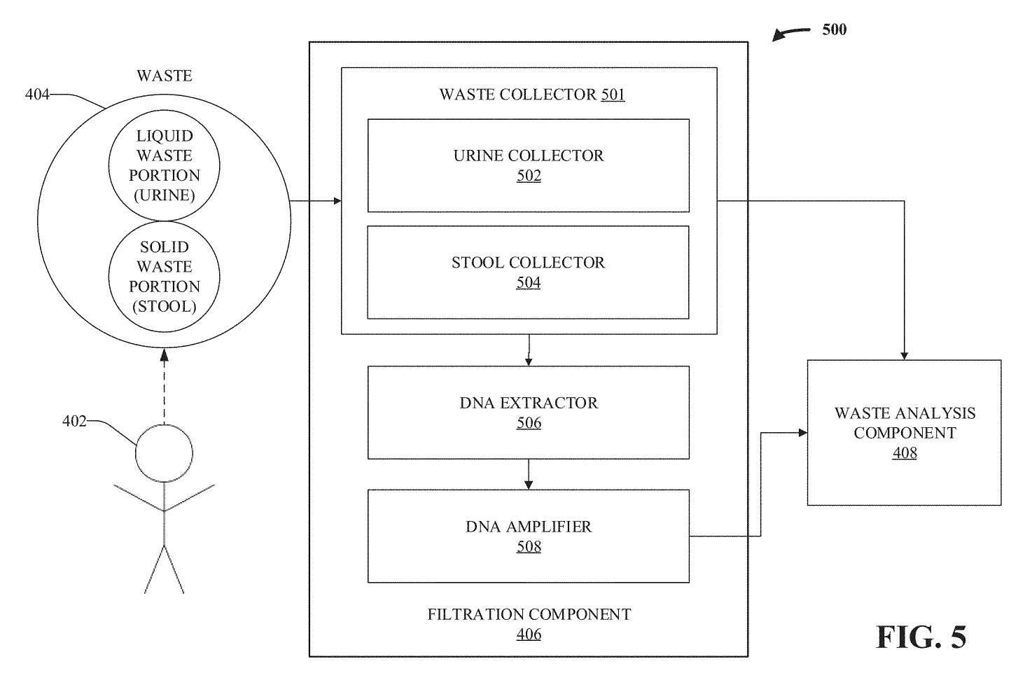

Now, consider FIG. 5. FIG. 5 illustrates a high-level functional block diagram of an example, nonlimiting filtration component in accordance with one or more embodiments disclosed herein. That is, FIG. 5 depicts a more detailed, non-limiting embodiment of the filtration component 406. Again, a user 402 can produce waste 404 (e.g., stool and/or urine), which can be deposited into the toilet bowl (not depicted) of the smart toilet system. In an embodiment, the filtration component 406 can comprise a waste collector 501 that can collect the waste 404 from the toilet bowl. As described above, the collection can occur actively (e.g., via actuated end-effectors, pumps, jets, and so on) and/or passively (e.g., a flushing operation of the toilet can cause waste 404 to flow into an intake orifice of the waste collector 501, and so on). In some cases, the waste collector 501 can include a urine collector 502, which can specifically collect urine (e.g., via a pump, a sieve, a grate, and so on) and/or a stool collector 504, which can specifically collect stool (e.g., via an actuated claw/surface, a sieve/net, and so on). In some embodiments, the waste collector 501 can simply include an inlet, a testing chamber, and an outlet, so as to collect a sample of waste 404 without separating and/or filtering the stool from the urine (e.g., omitting the specialized stool collector 504 and the specialized urine collector 502). As explained amply above, these collection components can function actively and/or passively. In an aspect, the waste collector 501 can comprise movable machinery and/or apparatuses which, in response to execution, can actively physically manipulate the waste 404. Such executable apparatuses can include, for example, robotic manipulators/arms, movable surfaces within the toilet bowl, water pumps/vacuums, air pumps/vacuums, and/or any other device which can touch, push, pull, move, grab, scoop, slice, and/or otherwise actively interact with the waste 404. In other embodiments, the waste collector 501 can comprise stationary apparatuses and/or fixtures which can passively collect the waste 404 as the waste enters the toilet bowl or as it is flushed down the toilet drain. Such passive fixtures can include, for example, grates, ledges, dividers, tubes, hooks, stationary claws, sieves, and so on. Those of skill in the art will appreciate that any other method and/or device for actively and/or passively collecting a waste sample can be incorporated into various embodiments of the disclosed innovation.

In one or more embodiments, after a sample is collected by the waste collector 501, at least some of the sample can be received by a DNA extractor 506. The DNA extractor 506 can extract the microbial DNA of the sample (e.g., break open the microbial cells in order to gain access to the biologically significant molecules within) via cellular disruption. As explained thoroughly above, cellular disruption can be accomplished through chemical lysis (e.g., by using lysis buffers, surfactants/detergents, lytic enzymes, chaotropic agents, and so on) and/or mechanical homogenization (e.g., bead beating, centrifugation, blending, microfluidizers, cryopulverization, and so on). Once lysed, the lysate can be refined by adding further surfactants/detergents, proteases, and RNase (e.g., to break down lipids, proteins, RNA, and other non-DNA molecules released by the cellular disruption). The lysate can then be purified by centrifugation to separate the microbial DNA from the broken down cellular material. As one of ordinary skill in the art will appreciate, any suitable method and/or apparatus for the automated extraction of DNA from a sample can be incorporated into the DNA extractor 506 in accordance with this disclosure. In some cases, inventories, reservoirs, and/or holding tanks/containers can be included in the DNA extractor 506 in order to store/hold and/or apply (e.g., via actuated valves) any required detergents, surfactants, lysis buffers, and so on.

In some embodiments, a flushing operation of the toilet can sufficiently physically homogenize a waste sample, thereby obviating the need for the DNA extractor 506.

After extraction/homogenization, the sample can, in some embodiments, be received by the DNA amplifier 508. The DNA amplifier 508 can, if needed, automatically amplify the target DNA in the extracted/homogenized sample by any suitable method and/or apparatus of amplification known in the art, such as basic PCR, emulsion PCR, bridge PCR, quantitative PCR, hot-start PCR, reverse transcription PCR (for the sequencing of RNA rather than DNA), long PCR, other known methods in the art, and so on. As explained above, basic PCR amplification can involve the use of a thermostable DNA polymerase, two oligonucleotide primers, deoxynucleotide triphosphates, a buffer solution, bivalent cations, monovalent cations, and a thermal cycler which cycles through the three acts of denaturation, primer annealing, and primer extension. One of ordinary skill in the art will appreciate that any suitable method and/or apparatus for automatically performing DNA amplification can be incorporated into the DNA amplifier 508 in various embodiments of the disclosed innovation.

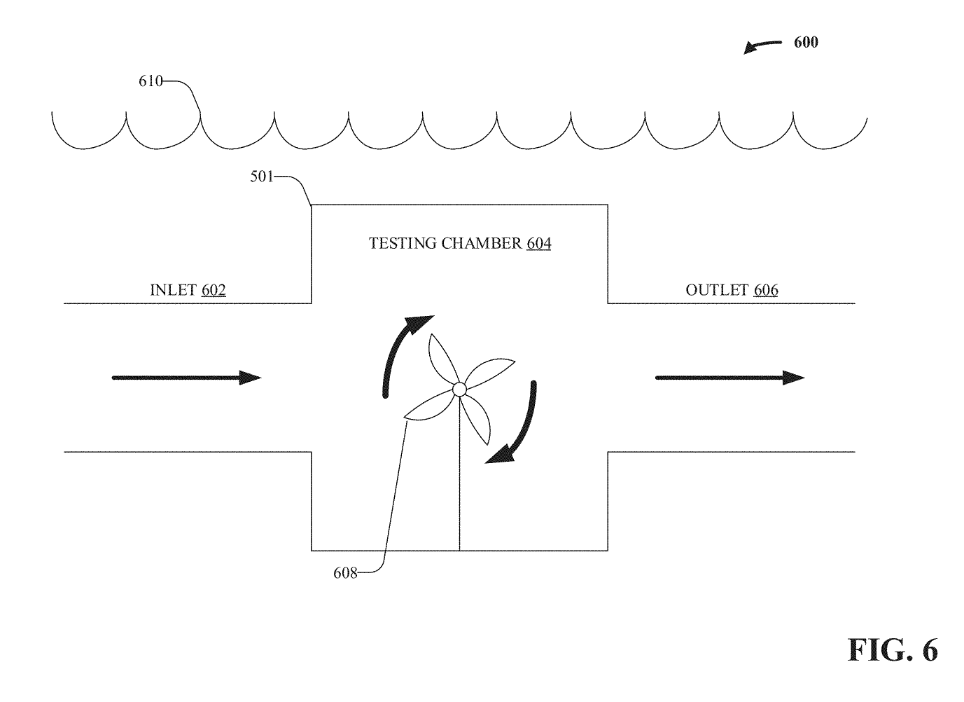

Now, consider FIG. 6. FIG. 6 illustrates a schematic of an example, nonlimiting waste collector in accordance one or more embodiments disclosed herein. That is, FIG. 6 depicts a non-limiting and high-level example of a potential structure of the waste collector 501.

As shown, the waste collector 501 can be fully and/or partially submerged in the toilet water 610 (e.g., by being affixed to and/or integrated into a side of the toilet bowl, and so on). In various embodiments, the waste collector 501 can include an inlet 602, a testing chamber 604, and an outlet 606. The inlet 602 can be any type of pipe, opening, aperture, and/or orifice that can receive a waste sample (e.g., toilet water mixed with the user's waste) from the toilet bowl. The testing chamber 604 can be a chamber and/or compartment that can receive the waste from the inlet 602. Moreover, medical and/or clinical tests, analyses, and/or examinations can be performed on the waste sample in the testing chamber 604. Thus, in some cases, any devices/apparatuses for performing DNA analysis and/or other waste analysis (e.g., DNA sequencer, image capture device, chemical test strip applicator, centrifuge, and so on) can be located in the testing chamber, thereby giving such devices access to the waste sample. In some cases, the DNA extractor 506 and/or the DNA amplifier 508 can also be located in the testing chamber (e.g., thereby having access to the sample). In some embodiments, the testing chamber 604 can include actuatable valves, walls, and/or separators (not depicted in FIG. 6) that can be used to retain the waste sample in the testing chamber 604 until the waste analyses are complete (e.g., via temporarily closing off and/or isolating the testing chamber 604 from the inlet 602 and the outlet 606, and so on). In other embodiments, such separators can be omitted. In one or more embodiments, the waste collector 501 can include a pump, propeller, and/or impeller 608, which can actively draw the toilet-water-and-waste mixture from the toilet bowl in through the inlet 602, into the testing chamber 604, and out through the outlet 606 (e.g., active waste collection). In some embodiments, the pump, propeller, and/or impeller 608 can be omitted, such that the toilet-water-and-waste mixture from the toilet bowl can passively flow into the inlet 602 and/or out the outlet 606 (e.g., a flushing operation of the toilet can cause the toilet water (and the waste in it) to swirl, thereby flowing through the inlet 602, into the testing chamber 604, and/or out the outlet 606). In some cases, the inlet 602 and the outlet 606 can be positioned on lateral surfaces of the waste collector 501 (e.g., such that a path from the inlet 602 to the outlet 606 can be substantially orthogonal to the radial direction of the toilet bowl), thereby increasing the amount of toilet water and waste that can flow into the testing chamber 604 during a flushing operation of the toilet.

As described above, the waste collector 501 can, in some embodiments, have specialized equipment/devices that are particularly suited to collecting particular types of waste (e.g., stool collector 504, urine collector 502, and so on). However, FIG. 6 depicts one or more embodiments in which the waste collector 501 does not contain specialized waste collection devices. Instead, any waste in the toilet bowl (e.g., stool, urine, and so on) can flow into the inlet 602, into the testing chamber 604, and out the outlet 606 in the embodiments shown in FIG. 6.

In one or more embodiments, after collection and/or preparation by the filtration component 406, the sample (e.g., the extracted sample from the DNA extractor 506, the amplified sample from the DNA amplifier 508, stool from the stool collector 504, and/or urine from the urine collector 502, and so on) can be received by the waste analysis component 408 for analysis (e.g., which can be inside and/or adjacent to the testing chamber 604.

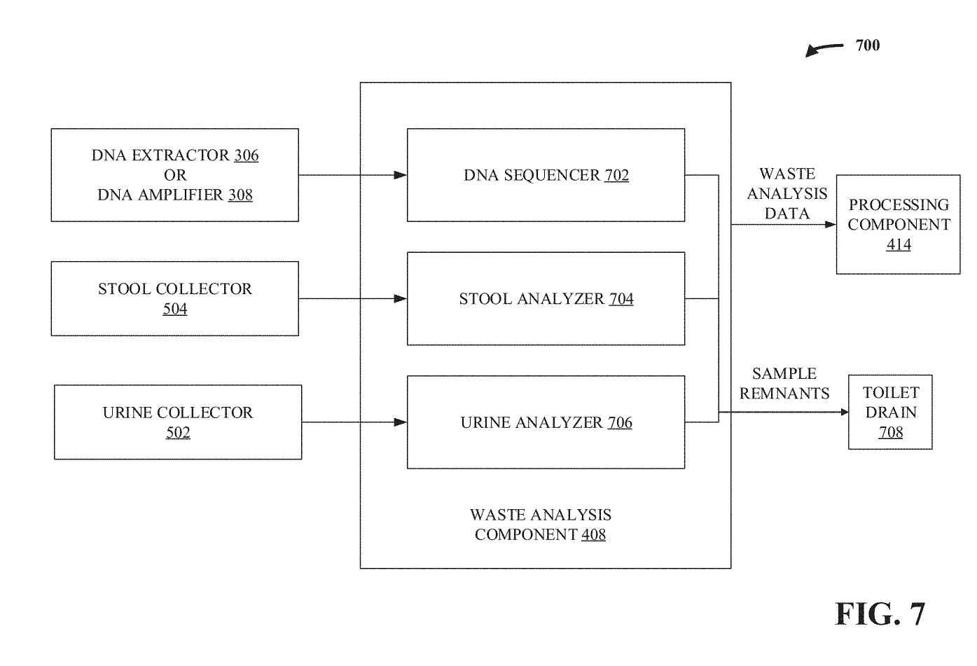

Now, consider FIG. 7. FIG. 7 illustrates a high-level functional block diagram of an example, nonlimiting waste analysis component in accordance with one or more embodiments disclosed herein. That is, FIG. 7 depicts a high-level and non-limiting example of the waste analysis component 408.

As shown, the waste analysis component 408 can, in some embodiments, comprise a DNA sequencer 702. The DNA sequencer 702 can receive an extracted, amplified, and/or raw sample from DNA extractor 506, from the DNA amplifier 508, and/or from the waste collector 501 (depending on whether a particular embodiment incorporates DNA extraction and/or amplification).

As explained thoroughly above, the DNA sequencer 702 can perform DNA sequencing analysis on at least part of the sample. In various embodiments, the DNA sequencer 702 can be configured to perform targeted amplicon sequencing (e.g., based on the 16S rRNA gene, and so on) and/or shotgun metagenomic sequencing, and so on. Furthermore, any DNA sequencing technology and/or platform can be utilized by the DNA sequencer 702. Thus, DNA sequencer 702 can comprise suitable hardware and/or software so as to implement any known technique of DNA sequencing, such as ion semiconductor sequencing, pyrosequencing, polymerase-based sequence-by-synthesis, ligation-based sequencing, single molecule real-time sequencing (also known as phospho-linked fluorescent nucleotide sequencing), dideoxy chain termination sequencing, nanopore sequencing, and so on. In some cases, the DNA sequencer 702 can include any automated DNA sequencing device known in the art. In some embodiments, the DNA sequencer 702, after performing the sequencing analysis, can produce at least one sequence read, which represents the order of the four nitrogenous bases in at least one DNA sequence identified in the sample, as described above. These sequence reads can then be processed/analyzed by the processing component 414.

In one or more embodiments, the waste analysis component 408 can comprise a stool analyzer 704 and/or a urine analyzer 706, which can perform stool analysis and/or urinalysis on the sample, respectively. As described above, the stool analyzer 704 and/or urine analyzer 706 can include any devices/apparatus for performing fecal pH tests, fecal fat tests, fecal occult blood tests, visual/microscopic examinations of stool/urine, chemical examinations of stool/urine, and so on (e.g., automated image capture devices, chemical/reagent test strip inventories and applicators, electronic noses, staining/dying devices, and so on). The results of such analyses can then be processed as described above in order to help determine the digestive health of the user.

Once the medical/clinical tests have been performed by the waste analysis component 408, any remnants of the sample can, in various embodiments, be discarded by being transported to the toilet drain 708 (e.g., flowing from the testing chamber 604, through the outlet 606, and back into the toilet bowl), as shown. In some cases, the raw data produced by the waste analysis component 408 (which can, in some cases, be categorized into DNA sequencing data from the DNA sequencer 702, stool analysis data from the stool analyzer 704, and/or urinalysis data from the urine analyzer 706) can be received by the processing component 414.

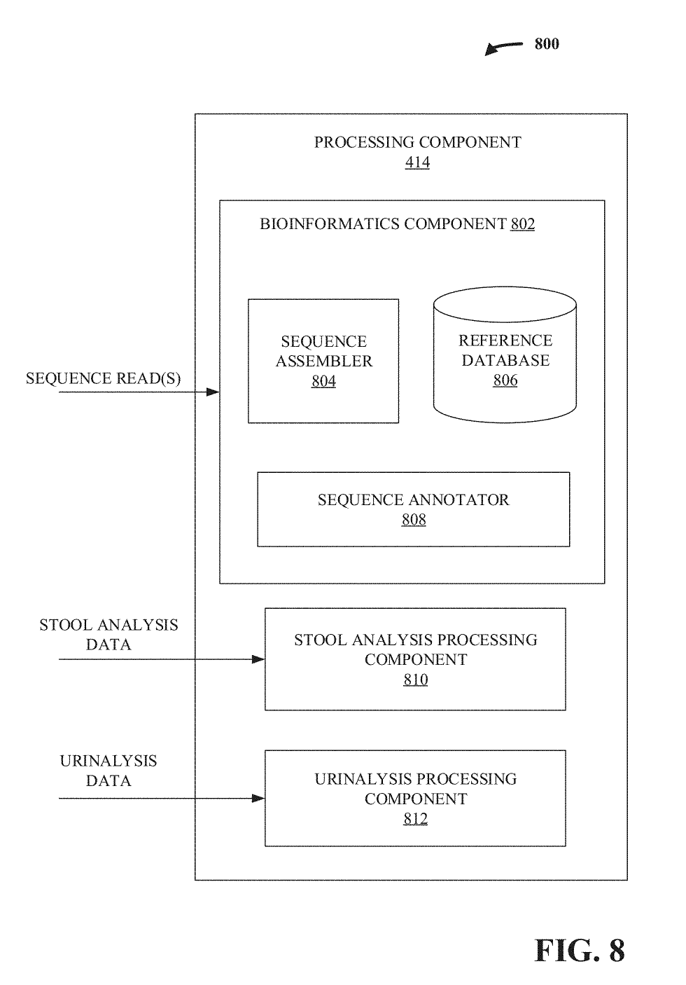

Now, consider FIG. 8. FIG. 8 illustrates a high-level functional block diagram of an example, nonlimiting processing component in accordance with one or more embodiments disclosed herein. That is, FIG. 8 depicts a high-level and non-limiting embodiment of the processing component 414.

As shown, the processing component 414 can, in one or more embodiments, comprise a bioinformatics component 802. The bioinformatics component 802 can receive the DNA sequencing data from the DNA sequencer 702 and then apply bioinformatics methods and techniques known in the art (e.g., described above) to convert the raw sequencing reads into medically significant information (e.g., the identities and quantities of microorganisms in the user's gut, and so on). As mentioned above, bioinformatics is a computational tool that leverages mathematics, statistics, chemistry, biology, and/or computer science in order to analyze vast amounts of data obtained through biological experiments. To this end, bioinformatics component 802 can, in some embodiments, comprise a sequence assembler 804, a reference database 806, and/or a sequence annotator 808.

The sequence assembler 804 can assemble the voluminous, fragmentary sequence reads produced by the DNA sequencer 702 to reconstruct at least one contiguous DNA sequence associated with the waste sample. To accomplish this, sequence assembler 804 can comprise any sequence assembly software and/or hardware known in the art, as explained above. Moreover, one having ordinary skill in the art will appreciate that the method of assembly employed by the sequence assembler 804 (e.g., de-novo assembly versus mapping assembly) is immaterial, as all such methods are in accordance with this disclosure.

Because some DNA sequencing techniques may not require assembly, some embodiments of the disclosed innovation can omit the sequence assembler 804.

The reference database 806, in some embodiments, can store known DNA sequences and/or other biologically significant information which can be leveraged to help convert the DNA sequencing data into medically significant results. For instance, after the sequence assembler 804 assembles at least one contiguous DNA sequence associated with the sample, the bioinformatics component 802 can computationally compare the obtained sequence to the collection of known sequences stored in the reference database 806. Once it is determined that a known DNA sequence sufficiently matches the obtained sequence, the microorganism to which the obtained DNA belongs will have been identified.

Although the reference database 806 is depicted in FIG. 8 as being local to the smart toilet system, one of ordinary skill in the art will appreciate that the reference database 806 may be located remotely and/or connected to the smart toilet system via a wired, wireless, network, and/or internet connection. Indeed, in various embodiments, the processing component 414 can be located remotely to the smart toilet system.

In addition to using the genetic information encoded in an obtained DNA sequence to identify the various microorganisms living in a user's gut, the bioinformatics component 802 can determine the relative population sizes of said microorganisms by comparing the relative amounts of the different DNA sequences identified in the sample with each other. In other words, the bioinformatics component 802 can use the genetic information contained within the obtained DNA sequences to identify which microorganisms are living in a user's gut and can then estimate the relative proportions of those various microorganisms in the user's gut by inferring that a microorganism which deposited more DNA in the waste sample is more populous than a microorganism that deposited less DNA in the waste sample. For example, if DNA sequencing analysis detects X units of DNA that matches the DNA of bacterium A and Y units of DNA that matches the DNA of bacterium B in the sample (where X>Y), the bioinformatics component 802 can infer both that the user's microflora includes bacterium A and bacterium B and that bacterium A is likely more populous in the user's gut than bacterium B by a factor of X to Y. Those of skill in the art will appreciate that other statistical, computational, and/or biological methods of estimating microflora population sizes are known in the art and are in accordance with this disclosure.

Now, if no known DNA sequences match an obtained DNA sequence, the sequence annotator 808 can, in some cases, structurally and/or functionally annotate the at least one obtained DNA sequence. As explained above, genetic annotation involves computationally identifying various genes and/or other coding regions in a DNA sequence and determining what these coding regions do (e.g., code for the production of a hazardous biological product, code for the performance of a certain biological function, and so on). This can be accomplished through any automatic annotation software/hardware tools known in the art. Thus, even if the bioinformatics component 802 is unable to identify a particular microorganism detected in a testable sample, it can, in some cases, determine, via sequence annotator 808, what the detected microorganism can do and can therefore classify the microorganism as beneficial, pathogenic, nonpathogenic, and so on.

In various other embodiments, the processing component 414 can comprise a stool analysis processing component 810 and/or a urinalysis processing component 812. As shown, these components can receive stool analysis data from the stool analyzer 704 and urinalysis data from the urine analyzer 706, respectively. Moreover, these components can perform any necessary and/or helpful post-test processing of raw data generated by the stool analyzer 704 and/or the urine analyzer 706, as explained above. For instance, the waste analysis component 408 can use automated imaging equipment to capture a microscopic image of a testable sample of stool and/or urine. Then, the processing component 414, via the stool analysis processing component 810 and/or the urinalysis processing component 812, can analyze the captured image by using suitable imaging and/or pattern recognition software in order to detect notable structures, such as parasites/ova, urinary casts, meat/vegetable fibers, red blood cells, white blood cells, epithelial cells, crystals, and so on. As another example, the waste analysis component 408 can use automated chemical/reagent test strips (e.g., dip sticks) to test the sample for certain chemical properties (e.g., pH, occult blood, proteins, glucose, ketones, and so on). As explained above, such chemical tests generally involve the use of chemically treated dip sticks which change color depending on the type and quantity of target chemicals in the sample. Thus, the processing component 414, via the stool analysis processing component 810 and/or the urinalysis processing component 812, can use suitable imaging and/or pattern recognition software to compare the colors yielded by the dip sticks to reference colors indicating known types and/or quantities of target chemicals. One with ordinary skill in the art will appreciate that any other type of processing known in the art that is required and/or helpful to convert the raw data produced by the waste analysis component 408 into medically significant results is in accordance with this disclosure and can accordingly be incorporated into the stool analysis processing component 810 and/or the urinalysis processing component 812. Through this processing, the presence and/or amounts of particular chemicals and/or structures, as well as other properties of interest of the sample, can be quantified. These quantified values can then be analyzed to help determine/evaluate the user's health.

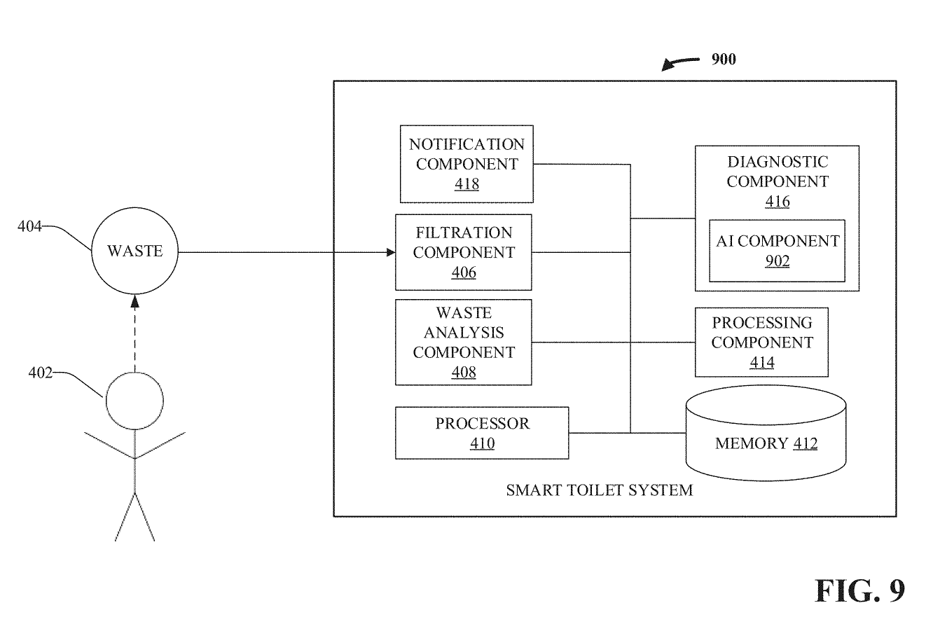

Now, consider FIG. 9. FIG. 9 illustrates a high-level functional block diagram of an example, nonlimiting smart toilet system comprising an artificial intelligence component in accordance with one or more embodiments disclosed herein.

As shown, the diagnostic component 416 can, in some embodiments, comprise an artificial intelligence (AI) component 902. As mentioned above, the AI component 902 can employ inferential logic, pattern recognition, and/or utility-based analysis in order to enhance the functionality and capabilities of the diagnostic component 416. In some cases, the AI component 902 can leverage a user's medical history, official diagnoses, complaints, symptoms, other idiosyncratic information, and/or other pertinent extrinsic information in order to help the diagnostic component 416 make appropriate diagnoses and/or recommendations (e.g., substantially as described above). For instance, the processing component 414 can produce data indicating that a user has an overabundance of microorganism X in his/her gut. The diagnostic component 416 can then determine that such an imbalance is caused by medical condition A, medical condition B, and so on. The user can then input pertinent facts regarding his/her medical history, symptoms, complaints, and so on, and said information can be leveraged by the AI component 902 to help narrow down the user's possible infirmities. Furthermore, the AI component 902 can, in some cases, use this additional/inputted information to help choose between recommended courses of action. For example, if the diagnostic component 416 determines that the best way to solve the detected imbalance is either through more consumption of food A or through vigorous exercise, the AI component 902 can leverage the user's age (and/or other inputted and/or learned information) in order to determine whether vigorous exercise would be appropriate (e.g., appropriate for young users, inappropriate for elderly users, and so on). On the other hand, if food A is known to be relatively indigestible for elderly people (which the diagnostic component 416 and/or the AI component 902 can learn via searching medical/clinical databases, and so on), the AI component 902 can determine that a modified course of action is more appropriate for an elderly user (e.g., some action besides eating food A and/or exercising). As another example, the user may input any medications he/she is currently taking. The AI component 902 can, in some cases, use that information to rule out recommended courses of action that are discouraged while on said medication.

One of ordinary skill in the art will appreciate that the AI component 902 can help to facilitate any and/or all of the higher-level functionalities previously disclosed above (e.g., learning the user's idiosyncratic health information from past waste analyses, applying idiosyncratic information to improve diagnoses and/or recommendations, monitoring the user's microflora profile over time to learn what recommendations work best for the user, explicit or implicit training, running simulations to test potential recommended courses of action, leveraging past analyses to improve current/future analyses and recommendations, piecing together a sequence of past microflora profiles to create a comprehensive and more accurate overall profile, and so on).

The embodiments of the present innovation herein can employ artificial intelligence (AI) to facilitate automating one or more features of the present innovation. The components can employ various AI-based schemes for carrying out various embodiments/examples disclosed herein. In order to provide for or aid in the numerous determinations (e.g., determine, ascertain, infer, calculate, predict, prognose, estimate, derive, forecast, detect, compute, and so on) of the present innovation, components of the present innovation can examine the entirety or a subset of the data to which it is granted access and can provide for reasoning about or determine states of the system, environment, and so on from a set of observations as captured via events and/or data. Determinations can be employed to identify a specific context or action, or can generate a probability distribution over states, for example. The determinations can be probabilistic; that is, the computation of a probability distribution over states of interest based on a consideration of data and events. Determinations can also refer to techniques employed for composing higher-level events from a set of events and/or data.

Such determinations can result in the construction of new events or actions from a set of observed events and/or stored event data, whether or not the events are correlated in close temporal proximity, and whether the events and data come from one or several event and data sources. Components disclosed herein can employ various classification (explicitly trained (e.g., via training data) as well as implicitly trained (e.g., via observing behavior, preferences, historical information, receiving extrinsic information, and so on)) schemes and/or systems (e.g., support vector machines, neural networks, expert systems, Bayesian belief networks, fuzzy logic, data fusion engines, and so on) in connection with performing automatic and/or determined action in connection with the claimed subject matter. Thus, classification schemes and/or systems can be used to automatically learn and perform a number of functions, actions, and/or determinations.

A classifier can map an input attribute vector, z=(z1, z2, z3, z4, zn), to a confidence that the input belongs to a class, as by f(z)=confidence(class). Such classification can employ a probabilistic and/or statistical-based analysis (e.g., factoring into the analysis utilities and costs) to determinate an action to be automatically performed. A support vector machine (SVM) can be an example of a classifier that can be employed. The SVM operates by finding a hyper-surface in the space of possible inputs, where the hyper-surface attempts to split the triggering criteria from the non-triggering events. Intuitively, this makes the classification correct for testing data that is near, but not identical to training data. Other directed and undirected model classification approaches include, e.g., naïve Bayes, Bayesian networks, decision trees, neural networks, fuzzy logic models, and/or probabilistic classification models providing different patterns of independence, any of which can be employed. Classification as used herein also is inclusive of statistical regression that is utilized to develop models of priority.

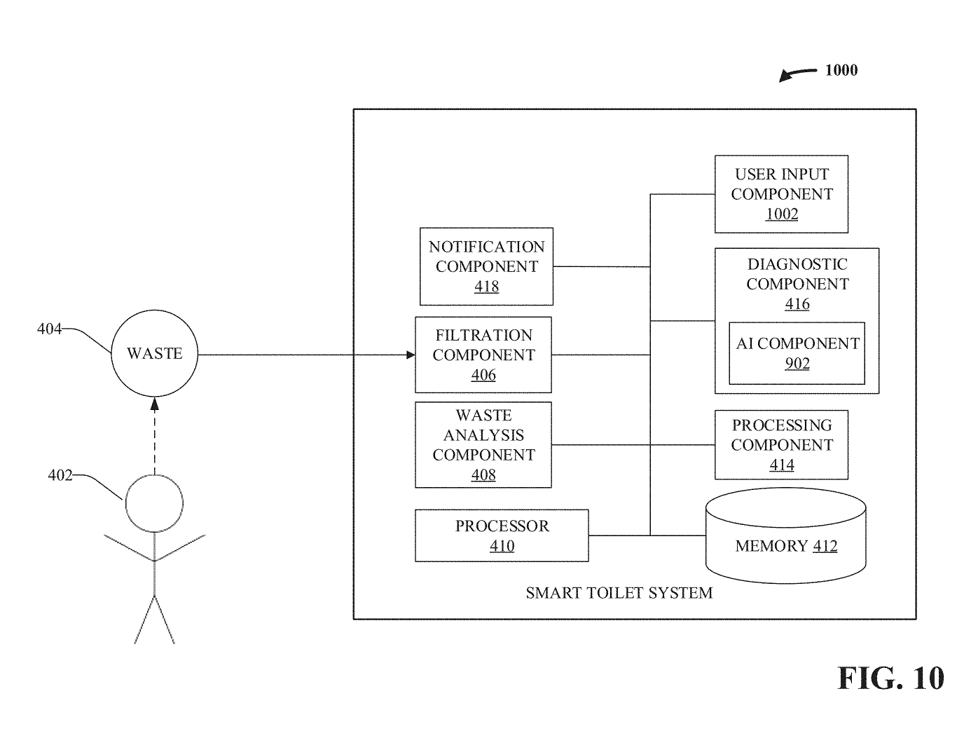

Now, consider FIG. 10. FIG. 10 illustrates a high-level functional block diagram of an example, nonlimiting smart toilet system comprising a user input component in accordance with one or more embodiments disclosed herein.

In some embodiments, the smart toilet system can comprise user input component 1002. As mentioned above, user input component 1002 can allow the user to make any pertinent, extrinsic information regarding the user (e.g., idiosyncratic information, preferences, age, weight, height, ethnicity, whether the user implemented past recommended courses of action, whether past recommended courses of action were effective, whether the user had visited medical professionals, and so on) available to the smart toilet system. Such information can include, for example, medical history, chronic ailments, pain/symptoms, medications, size, weight, height, BMI, gender, age, official diagnoses from medical professionals, whether the past recommendations helped the user's condition, and so on. Furthermore, the user input component 1002 can allow the user to change various settings of the smart toilet system, such as which type of medical analysis to run, which particular type of chemical/microorganism to look for, whether to run a waste analysis, and so on. One of ordinary skill in the art will appreciate that the user input component 1002 can be configured in any suitable way so as to allow the user to interact with the software and/or hardware of the smart toilet system (keyboard, keypad, buttons, touch screen, voice control, interface via a smart device of the user, and so on).

In some embodiments, the user input component 1002 can comprise any dedicated hardware that is connected, wired and/or wirelessly, to the smart toilet system. Such a device can be local and/or remote and can include, for example, computer screens and keypads/keyboards, touchscreens, buttons, joysticks, other human-interface devices, and so on. In other cases, user input component 1002 can comprise a wireless communication device that allows the smart toilet system to connect to a remote laptop computer, desktop computer, mobile device, PDA, tablet, and so on. One of ordinary skill in the art will appreciate that any other way known in the art of allowing the user to interact with the settings, software, and/or hardware of the smart toilet system can be implemented in various embodiments.

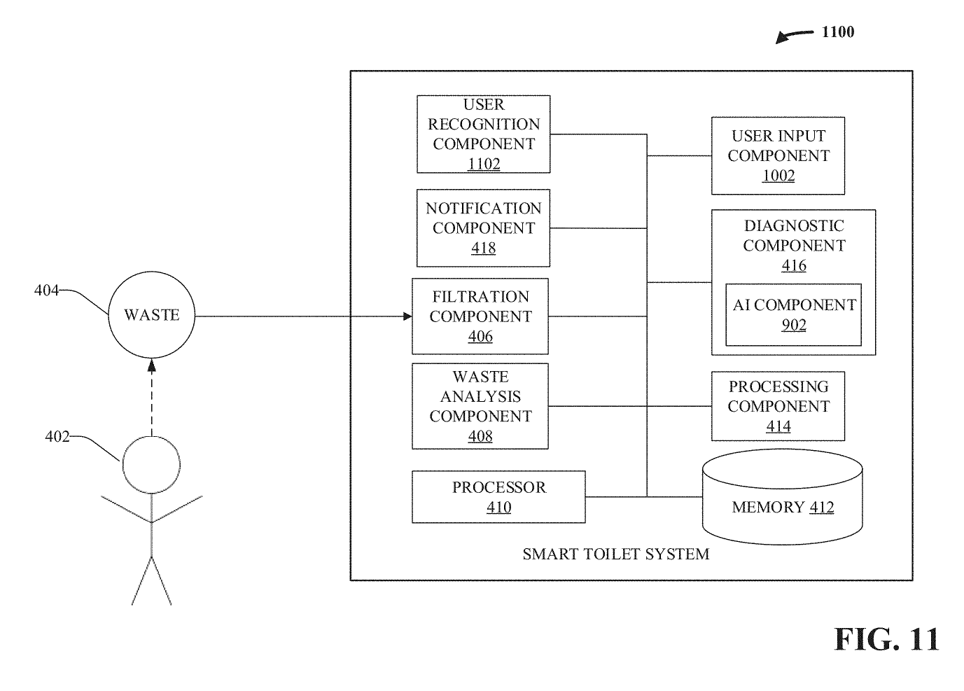

Now, consider FIG. 11. FIG. 11 illustrates a high-level functional block diagram of an example, nonlimiting smart toilet system comprising a user recognition component in accordance with one or more embodiments disclosed herein.

In various embodiments, the smart toilet system can comprise a user recognition component 1102. The user recognition component 1102 can automatically recognize the particular user using the smart toilet system (e.g., via voice recognition, weight recognition, appearance and/or image recognition, DNA recognition, and so on). Accordingly, the user recognition component 1102 can comprise weight scales (or any other suitable device known in the art for measuring weight), imaging equipment, and/or pattern recognition software so as to measure and recognize a user's weight, size, height, face, body, and so on. In some cases, the waste analysis component 408 can detect human DNA (e.g., a user's DNA) in the waste sample. Thus, the smart toilet system can recognize which user is currently using the smart toilet system by detecting the human DNA in the waste sample (e.g., user A is using the system if DNA of user A is detected, user B is using the system if DNA of user B is detected, and so on). Those of skill in the art will appreciate that any other method/device for automatically recognizing a particular user can be implemented in various embodiments.

Because the user recognition component 1102 can enable the smart toilet system to automatically detect and recognize a user, the smart toilet system can access previous settings, analysis results, medical histories, complaints, symptoms, diagnoses, recommended solutions, and so on that are associated with that user. The smart toilet system can then query the user to determine whether the previous diagnoses and/or recommended solutions proved helpful and/or beneficial or whether they were off the mark and/or ineffective. The smart toilet system can then take this information into account when making future diagnoses and/or recommendations (e.g. substantially as described above). For example, the smart toilet system can determine that a user has too much of microorganism X in his/her gut. The smart toilet system can then determine any medical conditions that are associated with an overabundance of microorganism X and can accordingly recommend one or more solutions so as to cure the imbalance. The next time the user uses the smart toilet system, the smart toilet system can then recognize the user, via the user recognition component 1102, and query the user whether he/she has improved and whether the previous diagnoses and/or recommendations proved useful/effective. If the answer is yes, the smart toilet system can note that said diagnosis and/or recommendation were effective. If the answer is no, the smart toilet system can leverage this information when making future diagnoses and/or recommendations (e.g., infer that similar recommendations would not be effective, and so on).

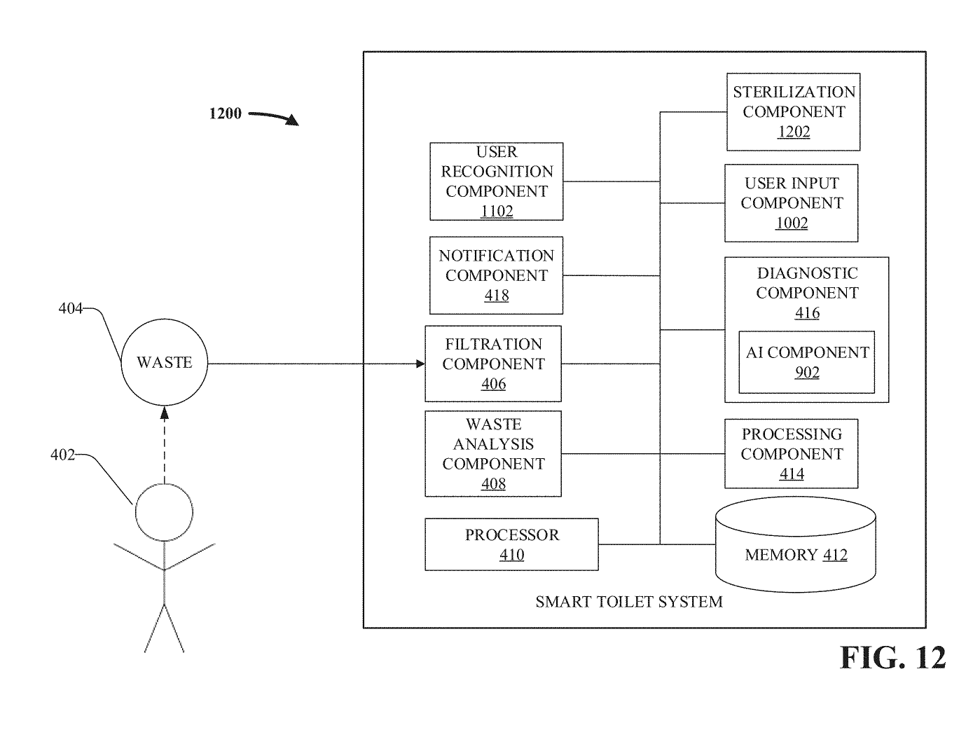

Now, consider FIG. 12. FIG. 12 illustrates a high-level functional block diagram of an example, nonlimiting smart toilet system comprising a sterilization component in accordance with one or more embodiments disclosed herein.

In various embodiments, the smart toilet system can comprise a sterilization component 1202. The sterilization component can, in some cases, automatically sterilize and/or otherwise clean the smart toilet system after and/or before analyzing a user's waste sample. This can allow the smart toilet system to analyze a waste sample from a different user with substantially reduced risk of cross-contaminating the first user's sample with the second user's sample.

In one or more embodiments, the sterilization component can include pumps, propellers, and/or jets that rinse the smart toilet system with one or more disinfectants (e.g., ethanol, alcohol, bleach, soap, and so on). For instance, the pumps/jets of the sterilization component 1202 can flood such cleaning agents on, in, and/or through the filtration component 406 and/or the waste analysis component 408 (e.g., the components that physically handle and/or come into contact with the user's waste), thereby removing any genetic material from a previous waste analysis so as to prevent contaminating a future waste analysis. In some cases, the smart toilet system can be cleansed/sanitized by simply applying conventional cleaning techniques to the toilet (e.g., filling the toilet bowl with a cleaning solution such that the smart toilet system uses a pump to cycle the cleaning solution through its contaminated components, and so on).

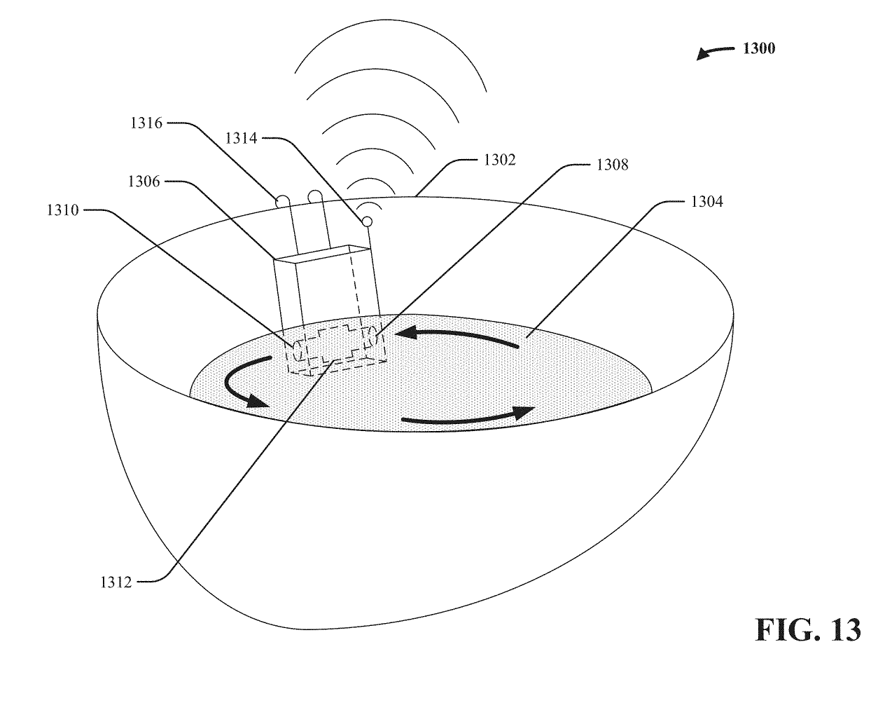

Now, consider FIG. 13. FIG. 13 illustrates a perspective schematic of an example, nonlimiting modular device affixable to a toilet bowl and that facilitates microbiome screening in accordance with one or more embodiments disclosed herein.

As thoroughly described above, various embodiments of the disclosed innovation can include physically integrating (e.g., manufacturing) DNA analysis technology (and/or other technology) into a standard toilet design, resulting in a single, integrated smart toilet having a toilet bowl and a toilet water reservoir, being connected to the plumbing of a building, and including waste analyzing technology. In one or more embodiments, however, the disclosed innovation can be embodied in a modular/portable device that can be affixed to the inside of a toilet bowl (e.g., via adhesives, screws, clamping devices, hanging devices, flotation devices, and so on), such that the device is at least partially submerged (or otherwise has physical access to) the toilet water in the toilet bowl. Once affixed to the toilet bowl, the device can perform any and/or all of the above-described waste analysis functionality (e.g., collecting and/or preparing a waste sample via a filtration component, analyzing the sample via a waste analysis component, processing the resulting raw data via a processing component, making diagnoses and/or recommendations via a diagnostic component, notifying the user of the results via a notification component, and so on). In some cases, the processing component, the diagnostic component, and/or the notification component (and so on) can be located remotely to the device (e.g., such that the device collects and analyzes a waste sample and subsequently transmits the resulting raw data to a remote computing platform (e.g., laptop, desktop, smart phone, the cloud, and so on) for computational processing). In such case, the device can include a transmitter, transceiver, and/or internet connector so as to transmit such data for processing.

To further understand such embodiments, consider FIG. 13. FIG. 13 depicts a toilet bowl 1302 with toilet water 1304. As shown, a housing 1306 can be affixed to the inside of the toilet bowl 1302 via the hanging wire(s) 1316. The housing 1306 can be at least partially submerged in the toilet water 1304, such that the toilet water 1304 can flow into and/or out of an inlet 1308 and/or an outlet 1310. In some cases, the inlet 1308 and outlet 1310 can be substantially like the inlet 602 and the outlet 606, described above. In some embodiments, the housing 1306 can further include a testing chamber 1312 (e.g., like the testing chamber 604 described above) that can receive a waste sample from the inlet 1308. As described above, one or more medical/clinical examinations can be performed on the waste sample in the testing chamber 1312. After such examinations, the waste sample can be expelled from the testing chamber 1312 to the outlet 1310.

In some embodiments, the housing can include a pump, propeller, and/or impeller (not depicted in FIG. 13) that can actively draw a waste sample in through the inlet 1308 and out through the outlet 1310. In some cases, the housing can passively collect a waste sample via a flushing operation of the toilet. As shown, a flushing operation of the toilet can induce a swirling motion in the toilet water 1304 (indicated by the cyclic arrows in FIG. 13). Such swirling motion can cause the toilet water (which can carry the user's waste) to flow into the inlet 1308, thereby facilitating passive automated collection of a waste sample.

To improve the ability of the device to collect the waste sample, the inlet 1308 and/or the outlet 1310 can be positioned on lateral and/or side surfaces of the housing 1306 (as shown in FIG. 13). In such case, a path of the waste sample from the inlet 1308, through the testing chamber 1312, and to the outlet 1310 can be substantially orthogonal to a radial direction of the toilet bowl 1302; that is, substantially parallel with the swirling motion of the toilet water 1304.

Those of skill in the art will appreciate that any of the above-described components (e.g., filtration component, waste analysis component, processing component, diagnostic component, notification component, AI component, user input component, user recognition component, sterilization component, and so on) can be incorporated into the housing 1306.

As shown, the housing 1306 can also include a transmitter/transceiver/antenna 1314. In such case, the device can collect and analyze a waste sample, and can subsequently transmit resulting raw data to a computing platform and/or the cloud for computational processing (as described above).

For simplicity of explanation, the computer-implemented methodologies are depicted and described as a series of acts. It is to be understood and appreciated that the subject innovation is not limited by the acts illustrated and/or by the order of acts. For example, acts can occur in various orders and/or concurrently, and with other acts not presented and described herein. Furthermore, not all illustrated acts can be required to implement the computer-implemented methodologies in accordance with the disclosed subject matter. In addition, those skilled in the art will understand and appreciate that the computer-implemented methodologies could alternatively be represented as a series of interrelated states via a state diagram or events. Additionally, it should be further appreciated that the computer-implemented methodologies disclosed hereinafter and throughout this specification are capable of being stored on an article of manufacture to facilitate transporting and transferring such computer-implemented methodologies to computers. The term article of manufacture, as used herein, is intended to encompass a computer program accessible from any computer-readable device or storage media.

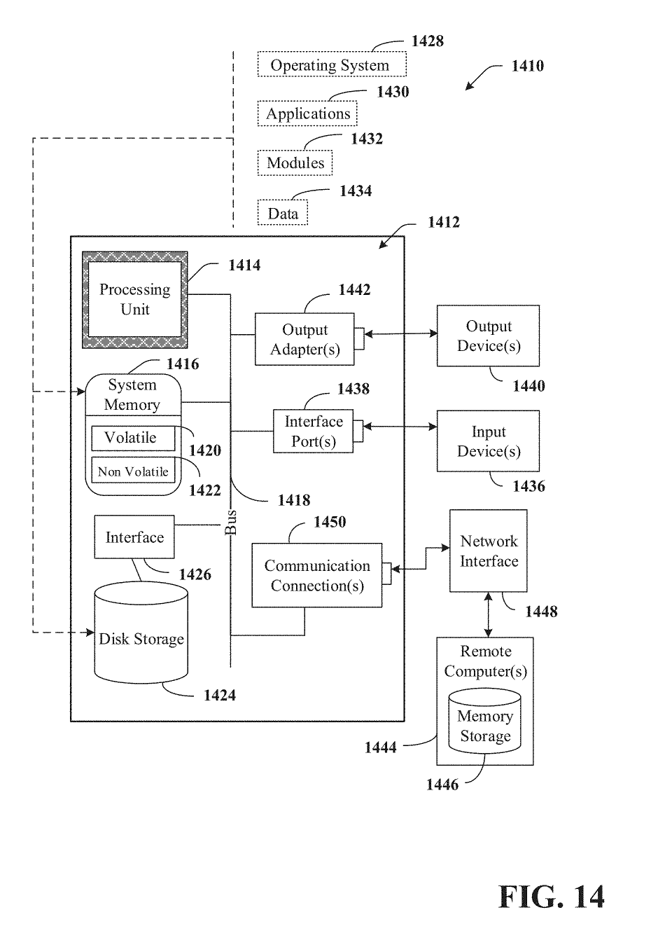

In order to provide a context for the various aspects of the disclosed subject matter, FIG. 14 as well as the following discussion are intended to provide a general description of a suitable environment in which the various aspects of the disclosed subject matter can be implemented. FIG. 14 illustrates a block diagram of an example, non-limiting operating environment in which one or more embodiments described herein can be facilitated. Repetitive description of like elements employed in other embodiments described herein is omitted for sake of brevity. With reference to FIG. 14, a suitable operating environment 1410 for implementing various aspects of this disclosure can also include a computer 1412. The computer 1412 can also include a processing unit 1414, a system memory 1416, and a system bus 1418. The system bus 1418 couples system components including, but not limited to, the system memory 1416 to the processing unit 1414. The processing unit 1414 can be any of various available processors. Dual microprocessors and other multiprocessor architectures also can be employed as the processing unit 1414. The system bus 1418 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Firewire (IEEE 1394), and Small Computer Systems Interface (SCSI). The system memory 1416 can also include volatile memory 1420 and nonvolatile memory 1422. The basic input/output system (BIOS), containing the basic routines to transfer information between elements within the computer 1412, such as during start-up, is stored in nonvolatile memory 1422. By way of illustration, and not limitation, nonvolatile memory 1422 can include read only memory (ROM), programmable ROM (PROM), electrically programmable ROM (EPROM), electrically erasable programmable ROM (EEPROM), flash memory, or nonvolatile random access memory (RAM) (e.g., ferroelectric RAM (FeRAM). Volatile memory 1420 can also include random access memory (RAM), which acts as external cache memory. By way of illustration and not limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM.

Computer 1412 can also include removable/non-removable, volatile/non-volatile computer storage media. FIG. 14 illustrates, for example, a disk storage 1424. Disk storage 1424 can also include, but is not limited to, devices like a magnetic disk drive, floppy disk drive, tape drive, Jaz drive, Zip drive, LS-100 drive, flash memory card, or memory stick. The disk storage 1424 also can include storage media separately or in combination with other storage media including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage 1424 to the system bus 1418, a removable or non-removable interface is typically used, such as interface 1426. FIG. 14 also depicts software that acts as an intermediary between users and the basic computer resources described in the suitable operating environment 1410. Such software can also include, for example, an operating system 1428. Operating system 1428, which can be stored on disk storage 1424, acts to control and allocate resources of the computer 1412. System applications 1430 take advantage of the management of resources by operating system 1428 through program modules 1432 and program data 1434, e.g., stored either in system memory 1416 or on disk storage 1424. It is to be appreciated that this disclosure can be implemented with various operating systems or combinations of operating systems. A user enters commands or information into the computer 1412 through input device(s) 1436. Input devices 1436 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1414 through the system bus 1418 via interface port(s) 1438. Interface port(s) 1438 include, for example, a serial port, a parallel port, a game port, and a universal serial bus (USB). Output device(s) 1440 use some of the same type of ports as input device(s) 1436. Thus, for example, a USB port can be used to provide input to computer 1412, and to output information from computer 1412 to an output device 1440. Output adapter 1442 is provided to illustrate that there are some output devices 1440 like monitors, speakers, and printers, among other output devices 1440, which require special adapters. The output adapters 1442 include, by way of illustration and not limitation, video and sound cards that provide a means of connection between the output device 1440 and the system bus 1418. It should be noted that other devices and/or systems of devices provide both input and output capabilities such as remote computer(s) 1444.

Computer 1412 can operate in a networked environment using logical connections to one or more remote computers, such as remote computer(s) 1444. The remote computer(s) 1444 can be a computer, a server, a router, a network PC, a workstation, a microprocessor based appliance, a peer device or other common network node and the like, and typically can also include many or all of the elements described relative to computer 1412. For purposes of brevity, only a memory storage device 1446 is illustrated with remote computer(s) 1444. Remote computer(s) 1444 is logically connected to computer 1412 through a network interface 1448 and then physically connected via communication connection 1450. Network interface 1448 encompasses wire and/or wireless communication networks such as local-area networks (LAN), wide-area networks (WAN), cellular networks, etc. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL). Communication connection(s) 1450 refers to the hardware/software employed to connect the network interface 1448 to the system bus 1418. While communication connection 1450 is shown for illustrative clarity inside computer 1412, it can also be external to computer 1412. The hardware/software for connection to the network interface 1448 can also include, for exemplary purposes only, internal and external technologies such as, modems including regular telephone grade modems, cable modems and DSL modems, ISDN adapters, and Ethernet cards.

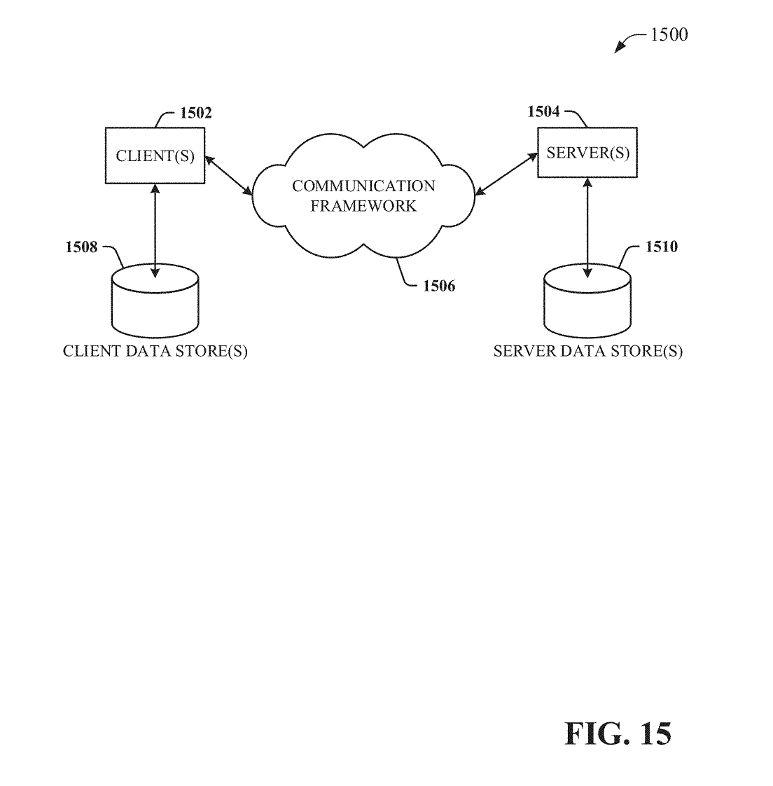

FIG. 15 is a schematic block diagram of a sample computing environment 1500 with which the disclosed subject matter can interact. The sample computing environment 1500 includes one or more client(s) 1502. The client(s) 1502 can be hardware and/or software (e.g., threads, processes, computing devices). The sample computing environment 1500 also includes one or more server(s) 1504. The server(s) 1504 can also be hardware and/or software (e.g., threads, processes, computing devices). The servers 1504 can house threads to perform transformations by employing one or more embodiments as described herein, for example. One possible communication between a client 1502 and a server 1504 can be in the form of a data packet adapted to be transmitted between two or more computer processes. The sample computing environment 1500 includes a communication framework 1506 that can be employed to facilitate communications between the client(s) 1502 and the server(s) 1504. The client(s) 1502 are operably connected to one or more client data store(s) 1508 that can be employed to store information local to the client(s) 1502. Similarly, the server(s) 1504 are operably connected to one or more server data store(s) 1510 that can be employed to store information local to the servers 1504.

The present innovation may be a system, a computer-implemented method, an apparatus and/or a computer program product at any possible technical detail level of integration. The computer program product can include a computer readable storage medium (or media) having computer readable program instructions thereon for causing a processor to carry out aspects of the present innovation. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of the present innovation can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present innovation.

Aspects of the present innovation are described herein with reference to flowchart illustrations and/or block diagrams of methods, apparatus (systems), and computer program products according to embodiments of the innovation. It will be understood that each block of the flowchart illustrations and/or block diagrams, and combinations of blocks in the flowchart illustrations and/or block diagrams, can be implemented by computer readable program instructions. These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, computer-implemented methods, and computer program products according to various embodiments of the present innovation. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on a computer and/or computers, those skilled in the art will recognize that this disclosure also can or can be implemented in combination with other program modules. Generally, program modules include routines, programs, components, data structures, etc. that perform particular tasks and/or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments where tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim. The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A smart-toilet system that facilitates automated microbiome screening, comprising:
a processor that executes computer-executable components stored on a computer-readable storage medium, the components comprising:
a filtration component that facilitates automated collection of a sample of biological waste of a user from a toilet;
a waste analysis component that performs an automated deoxyribonucleic acid (DNA) sequencing analysis on the sample to produce data characterizing the sample;
a processing component that applies bioinformatics to computationally process the data characterizing the sample;
a diagnostic component that, based on the processed data, diagnoses a potential malady afflicting the user and recommends a course of action to promote the user's health; and
a notification component that notifies the user of the diagnosis and recommendation.

2. The system of claim 1, wherein the filtration component comprises:
an inlet that receives the sample from the toilet;
a testing chamber that receives the sample from the inlet and in which the automated DNA sequencing analysis is performed on the sample; and
an outlet that receives the sample from the testing chamber and expels the sample back into the toilet.

3. The system of claim 2, wherein the sample is drawn through the inlet and expelled through the outlet by a pump or by a flushing operation of the toilet.

4. The system of claim 1, wherein the waste analysis component comprises a DNA sequencer that facilitates automated DNA sequencing of the sample, thereby producing at least one sequence read.

5. The system of claim 4, wherein the filtration component comprises a DNA extractor that facilitates physical or chemical cellular disruption of the sample prior to DNA sequencing.

6. The system of claim 5, wherein the filtration component further comprises a DNA amplifier that amplifies DNA extracted by the DNA extractor prior to DNA sequencing.

7. The system of claim 4, wherein the processing component comprises a bioinformatics component that assembles the at least one sequence read, thereby yielding at least one contiguous DNA sequence, and compares the at least one contiguous DNA sequence to known DNA sequences to identify a microflora organism in the user's gut and to which the at least one contiguous DNA sequence belongs.

8. The system of claim 7, wherein the bioinformatics component comprises:
a sequence assembler that assembles together the at least one sequence read into the at least one contiguous DNA sequence;
a local or remote reference database that stores known DNA sequences for comparison with the at least one contiguous DNA sequence; and
a sequence annotator that structurally or functionally annotates the at least one contiguous DNA sequence by comparing the at least one contiguous DNA sequence or portions thereof with the known DNA sequences or portions thereof.

9. The system of claim 7, wherein the bioinformatics component estimates a population size of the microflora organism in the user's gut and to which the at least one contiguous DNA sequence belongs based on a detected amount of the at least one contiguous DNA sequence, and wherein the diagnostic component leverages the estimated population size to diagnose the potential malady and recommend the course of action.

10. The system of claim 9, wherein the diagnostic component progressively constructs a microflora profile of the user's gut based on a plurality of analyses of samples of the user's waste over time by the smart toilet system.

11. The system of claim 1, wherein the waste analysis component comprises:
a chemical test strip applicator that exposes a chemical test strip to the sample; and
an image capture device that visually analyzes the exposed chemical test strip to identify target chemicals in the sample, and that facilitates a visual examination of the sample.

12. The system of claim 11, wherein the processing component utilizes image pattern recognition to process images captured by the image capture device, thereby identifying the target chemicals in the sample based on an image of the exposed chemical test strip, and identifying structures in the sample based on an image of the sample.

13. A computer-implemented method that facilitates automated microbiome screening, comprising:
executing, by a processor, computer-executable instructions to perform computer-executable acts, the acts comprising:
facilitating automated collection of a sample of biological waste of a user from a toilet;
performing an automated DNA sequencing analysis on the sample to produce data characterizing the sample;
computationally processing the data characterizing the sample via bioinformatics;
diagnosing, based on the processed data, a potential malady afflicting the user and recommending, based on the processed data, a course of action to promote the user's health; and
notifying the user of the diagnosis and recommendation.

14. The computer-implemented method of claim 13, wherein:
the facilitating automated collection of the sample comprises drawing the sample through an inlet and expelling the sample through an outlet via a pump or a flushing operation of the toilet.

15. The computer-implemented method of claim 13, wherein:
the performing an automated DNA sequencing analysis is accomplished via a DNA sequencer and produces at least one sequence read; and
the computationally processing the data comprises assembling, via a bioinformatics sequence assembler, the at least one sequence read into at least one contiguous DNA sequence, and comparing, via a bioinformatics sequence annotator, the at least one contiguous DNA sequence to known DNA sequences stored on a reference database to identify a microflora organism of the user's gut and to which the at least one contiguous DNA sequence belongs.

16. The computer-implemented method of claim 15, wherein:
the computationally processing the data further comprises estimating a population size of the microflora organism of the user's gut and to which the at least one contiguous DNA sequence belongs based on a detected amount of the at least one contiguous DNA sequence; and
the diagnosing a potential malady and recommending a course of action are based on the estimated population size.

17. The computer-implemented method of claim 16, further comprising progressively constructing a microflora profile of the user's gut based on a plurality of analyses of samples of the user's waste performed over time.

18. The computer-implemented method of claim 13, further comprising:
exposing, via a test strip applicator, a chemical test strip to the sample;
visually analyzing the chemical test strip via an image capture device; and
performing a visual examination of the sample via the image capture device.

19. A device for performing automated DNA analysis of biological waste, comprising:
a housing affixable to a toilet bowl;
an intake aperture in the housing and that receives a sample of biological waste from the toilet bowl during a flushing operation;
a DNA sequencer in the housing and that facilitates automated DNA sequencing of the sample, thereby yielding at least one sequence read; and
a transmitter in the housing and that transmits the at least one sequence read to a remote computing platform that performs bioinformatics processing of the at least one sequence read.

20. The device of claim 19, further comprising a DNA extractor in the housing and that facilitates physical or chemical cellular disruption of the sample prior to DNA sequencing.
